(19) United States
(12) Patent Application Publication
(10) Pub. No.: US 2025/0107434 A1
(43) Pub. Date: Mar. 27, 2025

(54) ORGANIC COMPOUNDS AND ORGANIC LIGHT-EMITTING DEVICE COMPRISING THE SAME

(71) Applicant: SFC CO., LTD., Cheongju-si (KR)

(72) Inventors: So-young SHIM, Cheongju-si (KR); Jin-hwi CHO, Cheongju-si (KR)

(73) Assignee: SFC CO., LTD., Cheongju-si (KR)

(21) Appl. No.: 18/884,430

(22) Filed: Sep. 13, 2024

(30) Foreign Application Priority Data
Sep. 14, 2023 (KR) 10-2023-0122753
Sep. 4, 2024 (KR) 10-2024-0119721

(51) Int. Cl. H10K 85/40; C07D 487/04; C07D 487/14; C07F 7/08; C07F 7/30; C09K 11/02; C09K 11/06; H10K 50/12; H10K 85/30; H10K 85/60; H10K 101/00; H10K 101/20

(52) U.S. Cl. CPC H10K 85/40; C07D 487/04; C07D 487/14; C07F 7/0812; C07F 7/30; C09K 11/02; C09K 11/06; H10K 85/30; H10K 85/6572; H10K 85/6574; H10K 85/658; C09K 2211/1007; C09K 2211/1018; H10K 50/121; H10K 2101/20; H10K 2101/27

(57) ABSTRACT

The present invention relates to an organic compound represented by the following [Formula 1], and a high-efficiency and long-lifetime organic light emitting device enabling significantly improved low voltage driving, and having excellent luminous efficiency, long lifetime and the like by employing the same as a light emitting layer host material in the device.

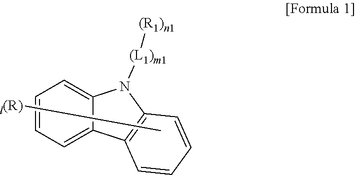

### TECHNICAL FIELD

The present invention relates to an organic compound employed in a light emitting layer of an organic light emitting device and an organic light emitting device including the organic compound.

### BACKGROUND ART

Organic light emitting devices are self-luminous devices in which electrons injected from an electron injecting electrode (cathode) recombine with holes injected from a hole injecting electrode (anode) in a light emitting layer to form excitons, which emit light while releasing energy. Such organic light emitting devices have the advantages of low driving voltage, high luminance, large viewing angle, and short response time and can be applied to full-color light emitting flat panel displays. Due to these advantages, organic light emitting devices have received attention as next-generation light sources.

The above characteristics of organic light emitting devices are achieved by structural optimization of organic layers of the devices and are supported by stable and efficient materials for the organic layers, such as hole injecting materials, hole transport materials, light emitting materials, electron transport materials, electron injecting materials, and electron blocking materials. However, more research still needs to be done to develop structurally optimized structures of organic layers for organic light emitting devices and stable and efficient materials for organic layers of organic light emitting devices.

Particularly, for maximum efficiency in a light emitting layer, an appropriate combination of energy band gaps of a host and a dopant is required such that holes and electrons migrate to the dopant through stable electrochemical paths to form excitons.

### DETAILED DESCRIPTION OF THE INVENTION

### Problems to be Solved by the Invention

Therefore, the present invention is intended to provide a host material in a light emitting layer having a characteristic structure, and a low-voltage, high-efficiency and long-lifetime organic light emitting device having significantly improved luminous efficiency and lifetime by including the same.

### Means for Solving the Problems

One aspect of the present invention provides an organic light emitting device including: a first electrode; a second electrode opposite to the first electrode; and an organic layer interposed between the first electrode and the second electrode, wherein the organic layer includes a light emitting layer including a host and a dopant, and the host includes at least one type of organic compound represented by the following [Formula 1].

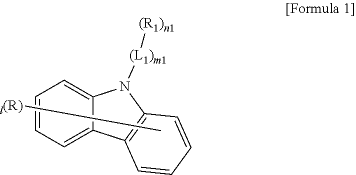

Specific structures of [Formula 1], specific compounds according to the present invention obtained therefrom, and a definition of each substituent will be described later.

### Effects of the Invention

The present invention relates to an organic compound employed in a light emitting layer in an organic light emitting device, and an organic light emitting device including the same. When the compound according to the present invention is employed as a host of a light emitting layer, an organic light emitting device with low voltage, high efficiency and long lifetime can be obtained, which is useful for not only lighting devices but also a variety of display devices such as flat panel displays, flexible displays, wearable displays, displays for automotives or aircraft, and displays for virtual or augmented reality.

### BEST MODE FOR CARRYING OUT THE INVENTION

Hereinafter, the present invention will be described in more detail.

One aspect of the present invention relates to an organic light emitting device including: a first electrode; a second electrode opposite to the first electrode; and an organic layer interposed between the first electrode and the second electrode, wherein the organic layer includes a light emitting layer including a host and a dopant, and the host includes at least one type of organic compound represented by the following [Formula 1].

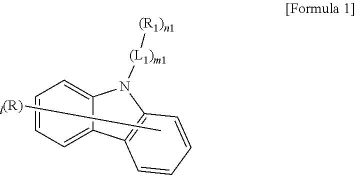

In [Formula 1],
L₁s are the same as or different from each other, and each independently a single bond, or any one selected from substituted or unsubstituted C₆-C₃₀ arylene, substituted or unsubstituted C₂-C₃₀ heteroarylene, and substituted or unsubstituted divalent cyclic groups in which a C₃-C₂₄ aliphatic ring and a C₅-C₂₄ aromatic ring are fused together.

R and R₁ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted cyclic groups in which a C₃-C₃₀ aliphatic ring and a C₅-C₃₀ aromatic ring are fused together, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano and halogen.

However, at least one of Rs of [Formula 1] is [Structural Formula 1].

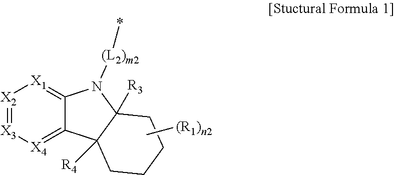

In [Structural Formula 1],
‘*’ means a bonding site linked to a carbon atom (C) in the carbazole of [Formula 1].

Herein, when L₂ in [Structural Formula 1] is a single bond, the carbon atom (C) of the carbazole in [Formula 1] and N linked to L₂ in [Structural Formula 1] are linked through a single bond; or,
when L₂ in the following [Structural Formula 1] is not a single bond, the carbon atom (C) of the carbazole in [Formula 1] and L₂ in [Structural Formula 1] are linked through a single bond.

X₁ to X₄ are the same as or different from each other and each independently N or CR₅, and at least one of X₁ to X₄ is N.

L₂s are the same as or different from each other, and each independently a single bond, or any one selected from substituted or unsubstituted C₆-C₃₀ arylene, substituted or unsubstituted C₂-C₃₀ heteroarylene, and substituted or unsubstituted divalent cyclic groups in which a C₃-C₂₄ aliphatic ring and a C₅-C₂₄ aromatic ring are fused together.

R₂ and R₅ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted cyclic groups in which a C₃-C₃₀ aliphatic ring and a C₅-C₃₀ aromatic ring are fused together, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano and halogen.

R₃ and R₄ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, and substituted or unsubstituted cyclic groups in which a C₃-C₃₀ aliphatic ring and a C₅-C₃₀ aromatic ring are fused together.

n₁ is an integer of 1 to 3, and when it is an integer of 2 or greater, R₁s are the same as or different from each other.

n₂ is an integer of 4, and R₂s are the same as or different from each other.

m₁ and m₂ are each independently an integer of 1 to 3, and when they are integers of 2 or greater, L₁s and L₂s are the same as or different from each other.

l is an integer of 8, and Rs except for the part linked to [Structural Formula 1] are the same as or different from each other.

The plurality of adjacent Rs and R₅s are optionally linked to each other to further form an alicyclic or aromatic monocyclic or polycyclic ring.

According to one embodiment of the present invention, at least one of R₁s in [Formula 1] may be any one selected from substituted or unsubstituted C₁-C₄₀ silyl and substituted or unsubstituted C₁-C₄₀ germanium.

According to one embodiment of the present invention, the rest except for the case in which any one of Rs in [Formula 1] is [Structural Formula 1] may be hydrogen or deuterium.

According to one embodiment of the present invention, m₁ in [Formula 1] is an integer of 1, and at the same time, L₁ may be substituted or unsubstituted C₆-C₃₀ arylene.

According to one embodiment of the present invention, R₂ to R₄ in [Structural Formula 1] are the same as or different from each other, and may be each independently any one selected from hydrogen, deuterium and substituted or unsubstituted C₁-C₁₀ alkyl.

According to one embodiment of the present invention, R₅ of CR₅ except for the case in which X₁ to X₄ in [Structural Formula 1] are N may be hydrogen or deuterium.

According to one embodiment of the present invention, m₂ in [Structural Formula 1] is an integer of 1, and at the same time, L₂ may be a single bond.

According to one embodiment of the present invention, at least one deuterium atom may substitute in [Formula 1].

Meanwhile, the term ‘substituted’ in the ‘substituted or unsubstituted’ in [Formula 1] and [Structural Formula 1] means being substituted with one or more substituents selected from the group consisting of deuterium, tritium, C₁-C₂₄ alkyl, C₁-C₂₄ haloalkyl, C₂-C₂₄ alkenyl, C₂-C₂₄ alkynyl, C₃-C₃₀ cycloalkyl, C₁-C₂₄ heteroalkyl, C₆-C₃₀ aryl, C₇-C₃₀ arylalkyl, C₇-C₃₀ alkylaryl, C₂-C₃₀ heteroaryl, C₂-C₃₀ heteroarylalkyl, cyclic groups in which a C₃-C₂₄ aliphatic ring and a C₅-C₂₄ aromatic ring are fused together, C₁-C₂₄ alkoxy, C₁-C₃₀ amine, C₁-C₃₀ silyl, C₁-C₃₀ germanium, C₆-C₂₄ aryloxy, C₆-C₂₄ arylthionyl, cyano, halogen, hydroxyl and nitro, and when there are two or more substituents, they are the same as or different from each other, and one or more hydrogen atoms in each of the substituents are optionally substituted with deuterium or tritium atoms.

In addition, another aspect of the present invention relates to an organic light emitting device including: a first electrode; a second electrode; and one or more organic layers interposed between the first electrode and the second electrode, wherein the compound represented by [Formula 1] defined above is included in the organic layer, preferably as a host of a light emitting layer including a host and a dopant.

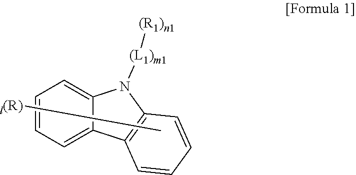

In addition, the light emitting layer has a structure formed with a host and a dopant, and the light emitting layer may further include a dopant material. Herein, the content of the dopant may be typically selected in a range of about 0.01 parts by weight to about 20 parts by weight based on about 100 parts by weight of the host, however, the content is not limited thereto.

In addition, the light emitting layer may further include various host and dopant materials in addition to the host and dopant compounds according to the present invention, and accordingly, in the light emitting layer, one or more types of compounds different from each other may be mixed or stacked and used as the dopant material as well as the host.

In addition, the organic compound represented by [Formula 1] used as the host is used as a blue phosphorescent host.

In addition, the organic layer of the organic light emitting device according to the present invention may be formed in a single layer structure, but may be formed in a multilayer structure in which two or more organic layers are stacked. For example, the organic layer may have a structure including a hole injecting layer, a hole transport layer, a hole blocking layer, a light emitting layer, an electron blocking layer, an electron transport layer, an electron injecting layer and the like. However, the structure is not limited thereto, and may also include a smaller or larger number of organic layers, and a preferred organic material layer structure of the organic light emitting device according to the present invention will be described in more detail in examples to be described later.

As one embodiment of the present invention, one or more types of host compounds different from the organic compound represented by [Formula 1] may be mixed and used, or stacked and used in addition to the organic compound represented by [Formula 1] as the host in the light emitting layer. In other words, in addition to one type of the compound represented by [Formula 1], one or more types of additional host compounds different therefrom may be further included as the host according to the present invention, and the two or more types of host compounds may be mixed or stacked and used. As for the stacking, the host compound different from the organic compound according to the present invention may be stacked and used in addition to the organic compound above or below the layer including the organic compound.

Herein, when two or more types of host compounds are mixed or stacked and used by further including one or more types of additional host compounds in addition to one type of the compound represented by [Formula 1], a compound having an electron acceptor moiety may be more preferably used as the additional host. High HOMO/LUMO levels of hole injection and electron injection barriers obtained by mixing or stacking the additional host with [Formula 1] that is an electron donor moiety having a carbazole group limit the recombination zone to the interface between the two hosts, and by the resulting advantage of minimizing a current loss and the like, a low-voltage, high-efficiency and long-lifetime organic light emitting device may be obtained.

Herein, the compound having an electron acceptor moiety is a compound having a moiety provided with an environment of readily accepting electrons from the outside in the molecule, such as an azine compound, a nitrogen-containing aromatic heterocyclic ring such as pyridine, pyrimidine or triazine, and a compound substituted with a cyano group (—CN), and may preferably include a compound including heteroaryl including 1 to 3 nitrogen (N) atoms in the molecule; or aryl including 1 to 3 cyano groups (CN) in the molecule.

More specifically, as the host in the light emitting layer of the organic light emitting device, one or more types of organic compounds represented by the following [Formula A] may be mixed and used in addition to the organic compound represented by [Formula 1], or an organic compound represented by the following [Formula A] may be stacked and used above or below the layer including the organic compound represented by [Formula 1].

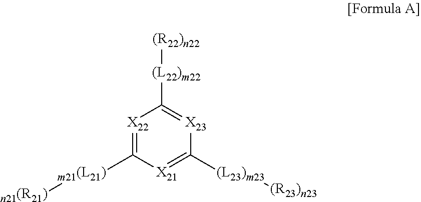

In [Formula A],
X₁ to X₃ are the same as or different from each other, and each independently N or CR₂₄, and at least one of X₂₁ to X₂₃ is N.

L₂₁ to L₂₃ are the same as or different from each other, and each independently any one selected from a single bond, substituted or unsubstituted C₆-C₃₀ arylene, substituted or unsubstituted C₂-C₃₀ heteroarylene, and substituted or unsubstituted divalent cyclic groups in which a C₃-C₂₄ aliphatic ring and a C₅-C₂₄ aromatic ring are fused together.

m₂₁ to m₂₃ are the same as or different from each other, and each independently an integer of 1 and 2, and when m₂₁ to m₂₃ are each an integer of 2, L₂ is to L₂₃s are the same as or different from each other.

R₂₁ to R₂₄ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted cyclic groups in which a C₃-C₃₀ aliphatic ring and a C₅-C₃₀ aromatic ring are fused together, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano and halogen.

The term ‘substituted’ in the ‘substituted or unsubstituted’ in [Formula A] means being substituted with one or more substituents selected from the group consisting of deuterium, tritium, C₁-C₂₄ alkyl, C₁-C₂₄ haloalkyl, C₂-C₂₄ alkenyl, C₂-C₂₄ alkynyl, C₃-C₃₀ cycloalkyl, C₁-C₂₄ heteroalkyl, C₆-C₃₀ aryl, C₇-C₃₀ arylalkyl, C₇-C₃₀ alkylaryl, C₂-C₃₀ heteroaryl, C₂-C₃₀ heteroarylalkyl, cyclic groups in which a C₃-C₂₄ aliphatic ring and a C₅-C₂₄ aromatic ring are fused together, C₁-C₂₄ alkoxy, C₁-C₃₀ amine, C₁-C₃₀ silyl, C₁-C₃₀ germanium, C₆-C₂₄ aryloxy, C₆-C₂₄ arylthionyl, cyano, halogen, hydroxyl and nitro, and when there are two or more substituents, they are the same as or different from each other, and one or more hydrogen atoms in each of the substituents are optionally substituted with deuterium or tritium atoms.

According to one embodiment of the present invention, X₂₁ to X₂₃ in [Formula A] are each N, and at least one of R₂₁ to R₂₃ may be substituted or unsubstituted carbazole.

In addition, according to one embodiment of the present invention, the dopant in the light emitting layer of the organic light emitting device may include at least one organometallic compound including a transition metal, and a polycyclic compound represented by the following [Formula 2] may be mixed or stacked and used in addition to the organometallic compound.

Accordingly, in the organic light emitting device according to one embodiment of the present invention, the light emitting layer may be formed by including a first host, a second host, an organometallic compound and a polycyclic compound of the following [Formula 2] (thermally activated delayed boron-based fluorescent material).

The organometallic compound functions as a sensitizer and the polycyclic compound functions as a light emitting dopant. The sensitizer compound may receive excitons from the first host and the second host and transfer the excitons to the light emitting dopant.

Accordingly, excitons are transferred from the sensitizer to the light emitting dopant compound through the Dexter energy transfer (DET) or Forster resonance transfer (FRET) mechanism, and the exciton energy transferred to the light emitting dopant compound may emit light while being is transferred to the ground state. Herein, the excitons of the sensitizer may be transferred from the first host and the second host by the FRET mechanism, or the generated excitons may be transferred from the host and formed by the DET mechanism.

In conclusion, the FRET or DET mechanism facilitates energy transfer between the sensitizer and the light emitting dopant and suppresses triplet-triplet annihilation, ensuring manufacture of high-efficiency of the organic light emitting device.

Thermally activated delayed boron-based fluorescent material that is the polycyclic compound represented by Formula 2 enables Forster energy transfer from the triplet of the phosphorescence sensitizer to the singlet of the thermally activated delayed boron-based fluorescent material, which may improve the lifetime by reducing the number of long-lived triplet excitons involved in the degradation of the device.

In addition, the rate of fluorescence resonance energy transfer from the phosphorescence sensitizer to the fluorescent material increases due to the high molar extinction coefficient, and the emission spectrum is narrowed by the multiple resonance effect, increasing color purity, and as a result of these effects, efficiency and lifetime of the device may be improved.

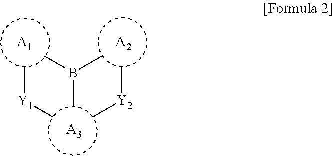

In [Formula 2],
Y₁ and Y₂ are the same as or different from each other, and each independently any one selected from O, S, NR₁₁, CR₁₂R₁₃, SiR₁₄R₁₅ and GeR₁₆R₁₇.

A₁ to A₃ are the same as or different from each other, and each independently any one selected from substituted or unsubstituted C₆-C₃₀ aromatic hydrocarbon rings, substituted or unsubstituted C₃-C₃₀ aliphatic hydrocarbon rings, substituted or unsubstituted C₂-C₃₀ aromatic heterocyclic rings, substituted or unsubstituted C₂-C₃₀ aliphatic heterocyclic rings, and substituted or unsubstituted cyclic groups in which a C₃-C₂₄ aliphatic ring and a C₅-C₂₄ aromatic ring are fused together.

R₁₁ to R₁₇ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted cyclic groups in which a C₃-C₃₀ aliphatic ring and a C₅-C₃₀ aromatic ring are fused together, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano and halogen.

Rn to R₁₇ are optionally linked to the rings A₁ to A₃ to further form an alicyclic or aromatic monocyclic or polycyclic ring.

R₁₂ and R₁₃, R₁₄ and R₁₅ and R₁₆ and R₁₇ are optionally linked to each other to further form an alicyclic or aromatic monocyclic or polycyclic ring.

The term ‘substituted’ in the ‘substituted or unsubstituted’ in [Formula 2] means being substituted with one or more substituents selected from the group consisting of deuterium, tritium, C₁-C₂₄ alkyl, C₁-C₂₄ haloalkyl, C₂-C₂₄ alkenyl, C₂-C₂₄ alkynyl, C₃-C₃₀ cycloalkyl, C₁-C₂₄ heteroalkyl, C₆-C₃₀ aryl, C₇-C₃₀ arylalkyl, C₇-C₃₀ alkylaryl, C₂-C₃₀ heteroaryl, C₂-C₃₀ heteroarylalkyl, cyclic groups in which a C₃-C₂₄ aliphatic ring and a C₅-C₂₄ aromatic ring are fused together, C₁-C₂₄ alkoxy, C₁-C₃₀ amine, C₁-C₃₀ silyl, C₁-C₃₀ germanium, C₆-C₂₄ aryloxy, C₆-C₂₄ arylthionyl, cyano, halogen, hydroxyl and nitro, and when there are two or more substituents, they are the same as or different from each other, and one or more hydrogen atoms in each of the substituents are optionally substituted with deuterium or tritium atoms.

According to one embodiment of the present invention, A₁ to A₃ are the same as or different from each other, and may be each independently any one selected from substituted or unsubstituted C₆-C₃₀ aromatic hydrocarbon rings, substituted or unsubstituted C₆-C₃₀ aromatic heterocyclic rings, and substituted or unsubstituted cyclic groups in which a C₃-C₂₄ aliphatic ring and a C₅-C₂₄ aromatic ring are fused together.

In the “substituted or unsubstituted C₁-C₃₀ alkyl”, “substituted or unsubstituted C₆-C₅₀ aryl” and the like in the present invention, the number of carbon atoms in the alkyl or aryl group indicates the number of carbon atoms constituting the unsubstituted alkyl or aryl moiety without considering the number of carbon atoms in the substituent(s). For example, a phenyl group substituted with a butyl group at the para-position corresponds to a C₆ aryl group substituted with a C₄ butyl group.

As used herein, the expression “optionally linked to each other or an adjacent group to form a ring” means that the corresponding adjacent substituents are bonded to each other or each of the corresponding substituents is bonded to an adjacent group to form a substituted or unsubstituted alicyclic or aromatic ring. The term “adjacent group” may mean a substituent on an atom directly attached to an atom substituted with the corresponding substituent, a substituent disposed sterically closest to the corresponding substituent or another substituent on an atom substituted with the corresponding substituent. For example, two substituents substituted at the ortho position of a benzene ring or two substituents on the same carbon in an aliphatic ring may be considered “adjacent” to each other. Optionally, the paired substituents each lose one hydrogen radical and are linked to each other to form a ring. The carbon atoms in the resulting alicyclic, aromatic mono- or polycyclic ring may be replaced by heteroatoms such as O, S, N, P, Si or Ge.

In the present invention, the alkyl groups may be straight or branched. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In the present invention, specific examples of the arylalkyl groups include, but are not limited to, phenylmethyl(benzyl), phenylethyl, phenylpropyl, naphthylmethyl, and naphthylethyl.

In the present invention, the haloalkyl means a substituent in which at least one hydrogen atom in alkyl as a substituent is substituted with halogen. The halogen may preferably be a fluorine atom, but is not limited thereto.

In the present invention, specific examples of the alkylaryl groups include, but are not limited to, tolyl, xylenyl, dimethylnaphthyl, t-butylphenyl, t-butylnaphthyl, and t-butylphenanthryl.

The alkenyl group is intended to include straight and branched ones and may be optionally substituted with one or more other substituents. The alkenyl group may be specifically a vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl) vinyl-1-yl, 2,2-bis(diphenyl-1-yl) vinyl-1-yl, stilbenyl or styrenyl group but is not limited thereto.

The alkynyl group is intended to include straight and branched ones and may be optionally substituted with one or more other substituents. The alkynyl group may be, for example, ethynyl or 2-propynyl but is not limited thereto.

The cycloalkenyl group is a non-aromatic cyclic unsaturated hydrocarbon group having one or more carbon-carbon double bonds. The cycloalkenyl group may be, for example, cyclopropenyl, cyclobutenyl, cyclopentenyl, cyclohexenyl, cycloheptenyl, 1,3-cyclohexadienyl, 1,4-cyclohexadienyl, 2,4-cycloheptadienyl or 1,5-cyclooctadienyl but is not limited thereto.

The aromatic hydrocarbon rings or aryl groups may be monocyclic or polycyclic ones. As used herein, the term “polycyclic” means that the aromatic hydrocarbon ring may be directly attached or fused to one or more other cyclic groups. The other cyclic groups may be aromatic hydrocarbon rings and other examples thereof include aliphatic heterocyclic rings, aliphatic hydrocarbon rings, and aromatic heterocyclic rings. Examples of the monocyclic aryl groups include, but are not limited to, phenyl, biphenyl, and terphenyl. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups but the scope of the present invention is not limited thereto.

The aromatic heterocyclic rings or heteroaryl groups refer to aromatic groups containing one or more heteroatoms such as O, S, N, P, Si, and Ge. Examples of the aromatic heterocyclic rings or heteroaryl groups include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, and phenothiazinyl groups.

The aliphatic hydrocarbon rings or cycloalkyl groups refer to non-aromatic rings consisting only of carbon and hydrogen atoms. The aliphatic hydrocarbon ring is intended to include monocyclic and polycyclic ones and may be optionally substituted with one or more other substituents. As used herein, the term “polycyclic” means that the aliphatic hydrocarbon ring may be directly attached or fused to one or more other cyclic groups. The other cyclic groups may be aliphatic hydrocarbon rings and other examples thereof include aliphatic heterocyclic, aryl, and heteroaryl groups. Specific examples of the aliphatic hydrocarbon rings include, but are not limited to, cycloalkyl groups such as cyclopropyl, cyclobutyl, cyclopentyl, adamantyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, and cyclooctyl, cycloalkanes such as cyclohexane and cyclopentane, and cycloalkenes such as cyclohexene and cyclobutene.

The aliphatic heterocyclic rings or heterocycloalkyl groups refer to aliphatic rings containing one or more heteroatoms such as O, S, Se, N, Si and Ge. The aliphatic heterocyclic ring is intended to include monocyclic or polycyclic ones and may be optionally substituted with one or more other substituents. As used herein, the term “polycyclic” means that the aliphatic heterocyclic ring such as heterocycloalkyl, heterocycloalkane or heterocycloalkene may be directly attached or fused to one or more other cyclic groups. The other cyclic groups may be aliphatic heterocyclic rings and other examples thereof include aliphatic hydrocarbon rings, aryl groups, and heteroaryl groups.

The cyclic groups in which an aliphatic ring and an aromatic ring are fused together refers to mixed aliphatic-aromatic cyclic groups in which at least one aliphatic ring and at least one aromatic ring are linked and fused together and which are overall non-aromatic. More specifically, the cyclic groups in which an aliphatic ring and an aromatic ring are fused together may be an aromatic hydrocarbon cyclic group fused with an aliphatic hydrocarbon ring, an aromatic hydrocarbon cyclic group fused with an aliphatic heterocyclic ring, an aromatic heterocyclic group fused with an aliphatic hydrocarbon ring, an aromatic heterocyclic group fused with an aliphatic heterocyclic ring, an aliphatic hydrocarbon cyclic group fused with an aromatic hydrocarbon ring, an aliphatic hydrocarbon cyclic group fused with an aromatic hydrocarbon ring, an aliphatic heterocyclic group fused with an aromatic hydrocarbon ring, and an aliphatic heterocyclic group fused with an aromatic heterocyclic ring. Specific examples of the cyclic groups in which an aliphatic ring and an aromatic ring are fused together include tetrahydronaphthyl, tetrahydrobenzocycloheptene, tetrahydrophenanthrene, tetrahydroanthracenyl, octahydrotriphenylene, tetrahydrobenzothiophene, tetrahydrobenzofuranyl, tetrahydrocarbazole, and tetrahydroquinoline. In addition, carbon in the cyclic groups in which an aliphatic ring and an aromatic ring are fused together may be replaced by heteroatoms such as O, S, N, P, Si or Ge.

The alkoxy group may be specifically a methoxy, ethoxy, propoxy, isobutyloxy, sec-butyloxy, pentyloxy, iso-amyloxy or hexyloxy group but is not limited thereto.

The silyl group is intended to include —SiH₃, alkylsilyl, arylsilyl, alkylarylsilyl, arylheteroarylsilyl, and heteroarylsilyl. The arylsilyl refers to a silyl group obtained by substituting one, two or three of the hydrogen atoms in —SiH₃ with aryl groups. The alkylsilyl refers to a silyl group obtained by substituting one, two or three of the hydrogen atoms in —SiH₃ with alkyl groups. The alkylarylsilyl refers to a silyl group obtained by substituting one of the hydrogen atoms in —SiH₃ with an alkyl group and the other two hydrogen atoms with aryl groups or substituting two of the hydrogen atoms in —SiH₃ with alkyl groups and the remaining hydrogen atom with an aryl group. The arylheteroarylsilyl refers to a silyl group obtained by substituting one of the hydrogen atoms in —SiH₃ with an aryl group and the other two hydrogen atoms with heteroaryl groups or substituting two of the hydrogen atoms in —SiH₃ with aryl groups and the remaining hydrogen atom with a heteroaryl group. The heteroarylsilyl refers to a silyl group obtained by substituting one, two or three of the hydrogen atoms in —SiH₃ with heteroaryl groups. The arylsilyl group may be, for example, substituted or unsubstituted monoarylsilyl, substituted or unsubstituted diarylsilyl, or substituted or unsubstituted triarylsilyl. The same applies to the alkylsilyl and heteroarylsilyl groups.

Each of the aryl groups in the arylsilyl, heteroarylsilyl, and arylheteroarylsilyl groups may be a monocyclic or polycyclic one. Each of the heteroaryl groups in the arylsilyl, heteroarylsilyl, and arylheteroarylsilyl groups may be a monocyclic or polycyclic one.

Specific examples of the silyl groups include trimethylsilyl, triethylsilyl, triphenylsilyl, trimethoxysilyl, dimethoxyphenylsilyl, diphenylmethylsilyl, diphenylvinylsilyl, methylcyclobutylsilyl, and dimethylfurylsilyl. One or more of the hydrogen atoms in each of the silyl groups may be substituted with the substituents mentioned in the aryl groups.

The amine group is intended to include —NH₂, alkylamine, arylamine, arylheteroarylamine, and heteroarylamine. The arylamine refers to an amine group obtained by substituting one or two of the hydrogen atoms in —NH₂ with aryl groups. The alkylamine refers to an amine group obtained by substituting one or two of the hydrogen atoms in —NH₂ with alkyl groups. The alkylarylamine refers to an amine group obtained by substituting one of the hydrogen atoms in —NH₂ with an alkyl group and the other hydrogen atom with an aryl group. The arylheteroarylamine refers to an amine group obtained by substituting one of the hydrogen atoms in —NH₂ with an aryl group and the other hydrogen atom with a heteroaryl group. The heteroarylamine refers to an amine group obtained by substituting one or two of the hydrogen atoms in —NH₂ with heteroaryl groups. The arylamine may be, for example, substituted or unsubstituted monoarylamine, substituted or unsubstituted diarylamine, or substituted or unsubstituted triarylamine. The same applies to the alkylamine and heteroarylamine groups.

Each of the aryl groups in the arylamine, heteroarylamine, and arylheteroarylamine groups may be a monocyclic or polycyclic one. Each of the heteroaryl groups in the arylamine, heteroarylamine, and arylheteroarylamine groups may be a monocyclic or polycyclic one.

The germanium group is intended to include —GeH₃, alkylgermanium, arylgermanium, heteroarylgermanium, alkylarylgermanium, alkylheteroarylgermanium, and arylheteroarylgermanium. The definitions of the substituents in the germanium groups follow those described for the silyl groups, except that the silicon (Si) atom in each silyl group is changed to a germanium (Ge) atom.

Specific examples of the germanium groups include trimethylgermane, triethylgermane, triphenyl germane, trimethoxygermane, dimethoxyphenylgermane, diphenylmethylgermane, diphenylvinylgermane, methylcyclobutylgermane, and dimethylfurylgermane. One or more of the hydrogen atoms in each of the germanium groups may be substituted with the substituents mentioned in the aryl groups.

The cycloalkyl, aryl, and heteroaryl groups in the cycloalkyloxy, aryloxy, heteroaryloxy, cycloalkylthioxy, arylthioxy, and heteroarylthioxy groups are the same as those exemplified above. Specific examples of the aryloxy groups include, but are not limited to, phenoxy, p-tolyloxy, m-tolyloxy, 3,5-dimethylphenoxy, 2,4,6-trimethylphenoxy, p-tert-butylphenoxy, 3-biphenyloxy, 4-biphenyloxy, 1-naphthyloxy, 2-naphthyloxy, 4-methyl-1-naphthyloxy, 5-methyl-2-naphthyloxy, 1-anthryloxy, 2-anthryloxy, 9-anthryloxy, 1-phenanthryloxy, 3-phenanthryloxy, and 9-phenanthryloxy groups. Specific examples of the arylthioxy groups include, but are not limited to, phenylthioxy, 2-methylphenylthioxy, and 4-tert-butylphenylthioxy groups.

The halogen group may be, for example, fluorine, chlorine, bromine or iodine.

According to one embodiment of the present invention, the compound represented by [Formula 1] may be any one selected from compounds represented by the following formulae, but is not limited thereto.

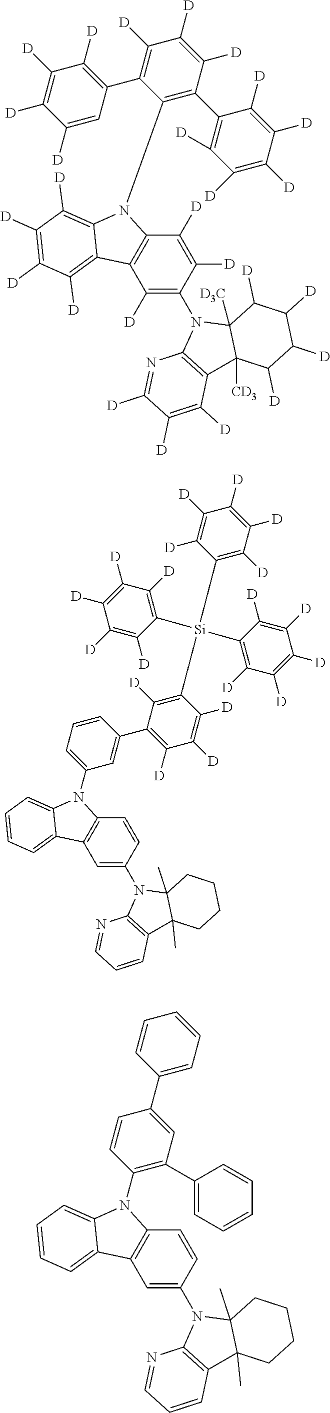

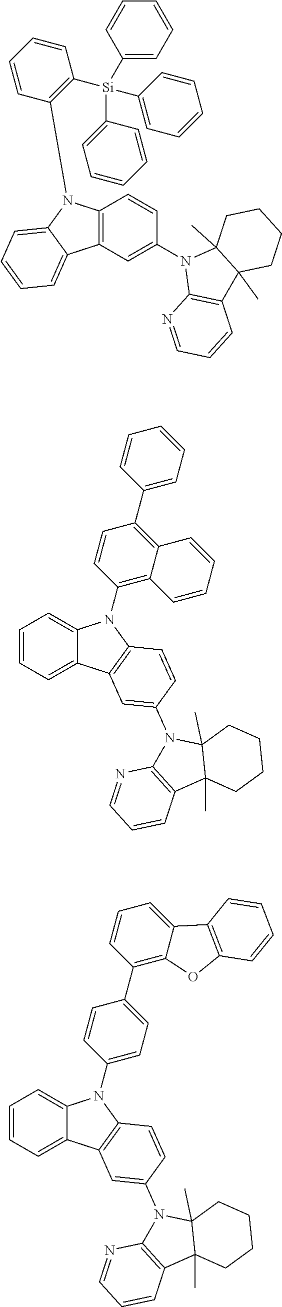 [SMILES: CC12CCCCC1(C)N(c1ccc3c(c1)c1ccccc1n3-c1ccccc1[Si](c1ccccc1)(c1ccccc1)c1ccccc1)c1ncccc12 ; CC12CCCCC1(C)N(c1ccc3c(c1)c1ccccc1n3-c1ccc(-c3ccccc3)c3ccccc13)c1ncccc12 ; CC12CCCCC1(C)N(c1ccc3c(c1)c1ccccc1n3-c1ccc(-c3cccc4c3oc3ccccc34)cc1)c1ncccc12]

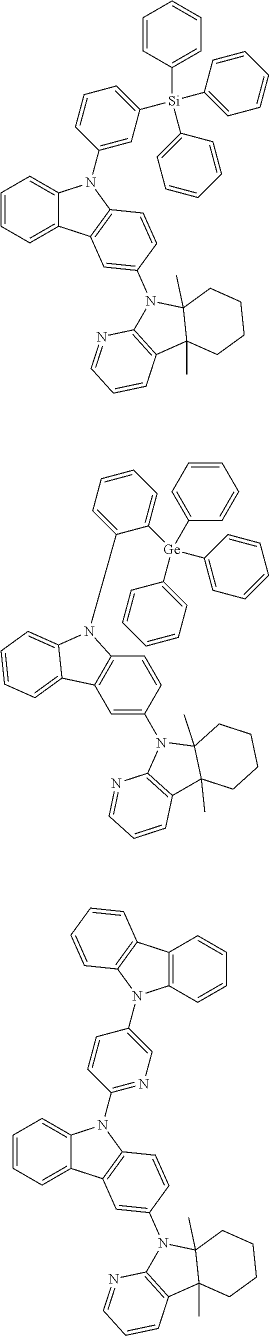

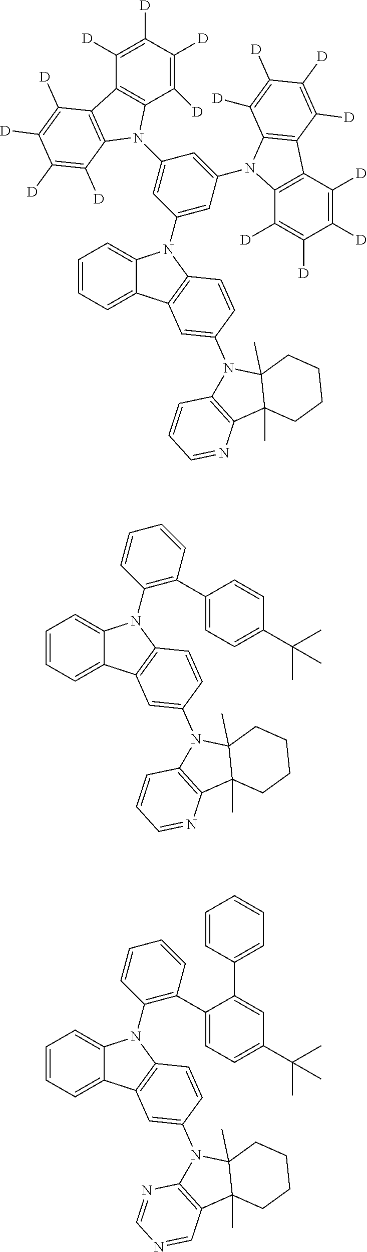 [SMILES: [2H]c1c([2H])c([2H])c2c(c1[2H])c1c([2H])c([2H])c([2H])c([2H])c1n2-c1cc(-n2c3ccccc3c3cc(N4c5cccnc5C5(C)CCCCC45C)ccc32)cc(-n2c3c([2H])c([2H])c([2H])c([2H])c3c3c([2H])c([2H])c([2H])c([2H])c32)c1 ; CC(C)(C)c1ccc(-c2ccccc2-n2c3ccccc3c3cc(N4c5cccnc5C5(C)CCCCC45C)ccc32)cc1 ; CC(C)(C)c1ccc(-c2ccccc2-n2c3ccccc3c3cc(N4c5ncncc5C5(C)CCCCC45C)ccc32)c(-c2ccccc2)c1]

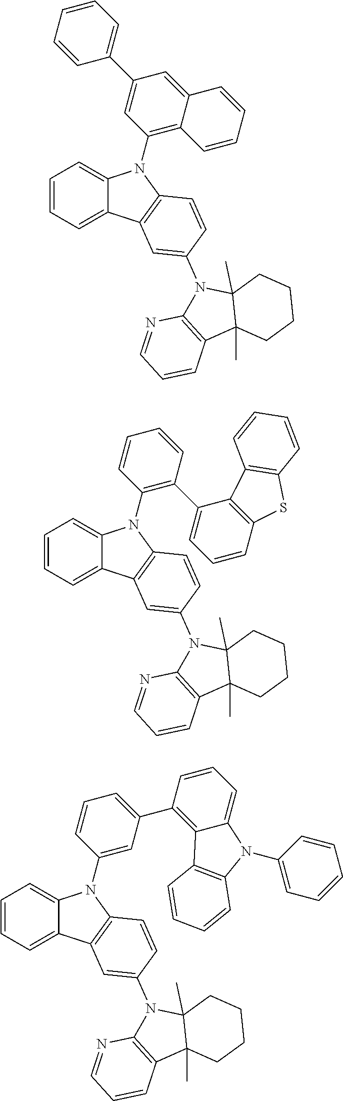

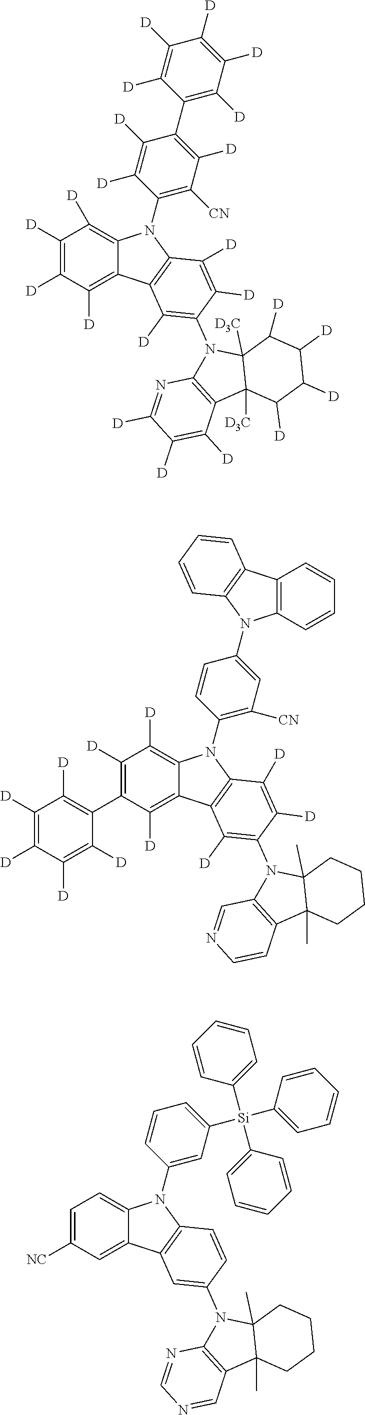 [SMILES: [2H]c1nc2c(c([2H])c1[2H])C1(C([2H])([2H])[2H])C([2H])C([2H])C([2H])C([2H])C1(C([2H])([2H])[2H])N2c1c([2H])c([2H])c2c(c1[2H])c1c([2H])c([2H])c([2H])c([2H])c1n2-c1c([2H])c([2H])c(-c2c([2H])c([2H])c([2H])c([2H])c2[2H])c([2H])c1C#N ; [2H]c1c([2H])c([2H])c(-c2c([2H])c([2H])c3c(c2[2H])c2c([2H])c(N4c5cnccc5C5(C)CCCCC45C)c([2H])c([2H])c2n3-c2ccc(-n3c4ccccc4c4ccccc43)cc2C#N)c([2H])c1[2H] ; CC12CCCCC1(C)N(c1ccc3c(c1)c1cc(C#N)ccc1n3-c1cccc([Si](c3ccccc3)(c3ccccc3)c3ccccc3)c1)c1ncncc12]

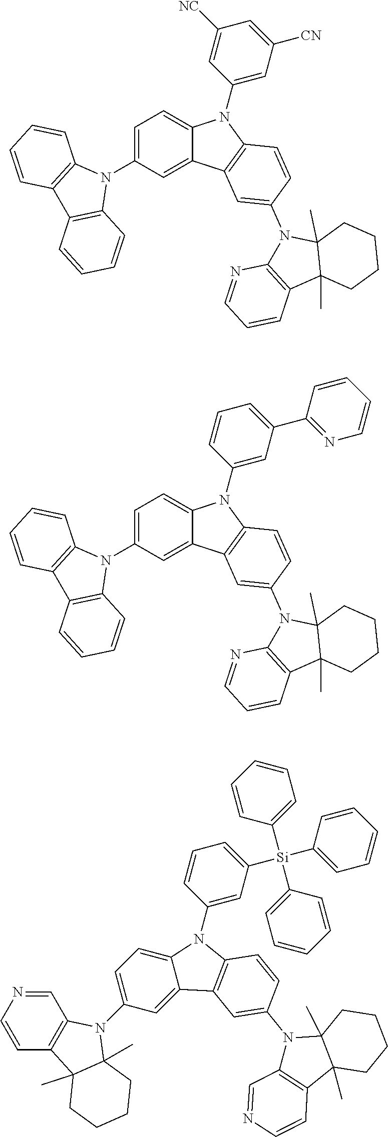 [SMILES: CC12CCCCC1(C)N(c1ccc3c(c1)c1cc(-n4c5ccccc5c5ccccc54)ccc1n3-c1cc(C#N)cc(C#N)c1)c1ncccc12 ; CC12CCCCC1(C)N(c1ccc3c(c1)c1cc(-n4c5ccccc5c5ccccc54)ccc1n3-c1cccc(-c3ccccn3)c1)c1ncccc12 ; CC12CCCCC1(C)N(c1ccc3c(c1)c1cc(N4c5cnccc5C5(C)CCCCC45C)ccc1n3-c1cccc([Si](c3ccccc3)(c3ccccc3)c3ccccc3)c1)c1cnccc12]

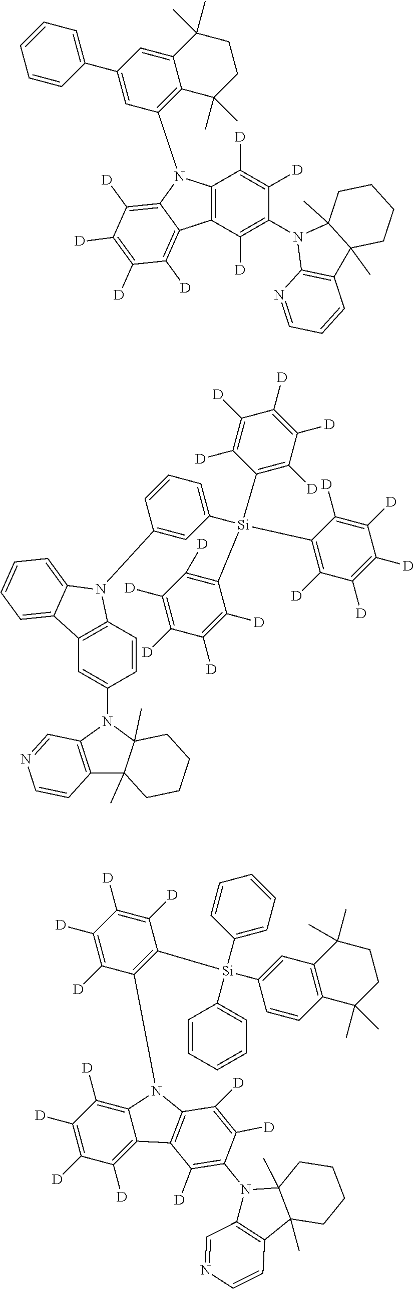 [SMILES: [2H]c1c([2H])c([2H])c2c(c1[2H])c1c([2H])c(N3c4ncccc4C4(C)CCCCC34C)c([2H])c([2H])c1n2-c1cc(-c2ccccc2)cc2c1C(C)(C)CCC2(C)C ; [2H]c1c([2H])c([2H])c([Si](c2cccc(-n3c4ccccc4c4cc(N5c6cnccc6C6(C)CCCCC56C)ccc43)c2)(c2c([2H])c([2H])c([2H])c([2H])c2[2H])c2c([2H])c([2H])c([2H])c([2H])c2[2H])c([2H])c1[2H] ; [2H]c1c([2H])c([2H])c([Si](c2ccccc2)(c2ccccc2)c2ccc3c(c2)C(C)(C)CCC3(C)C)c(-n2c3c([2H])c([2H])c([2H])c([2H])c3c3c([2H])c(N4c5cnccc5C5(C)CCCCC45C)c([2H])c([2H])c32)c1[2H]]

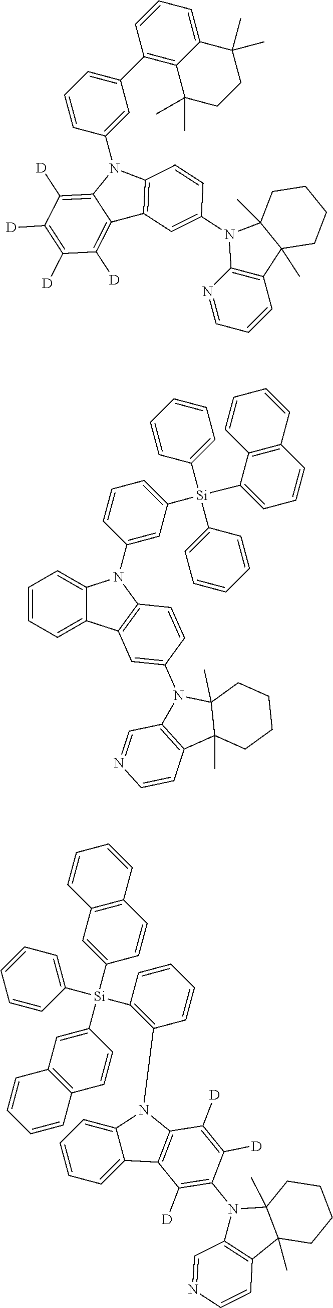 [SMILES: [2H]c1c([2H])c([2H])c2c(c1[2H])c1cc(N3c4ncccc4C4(C)CCCCC34C)ccc1n2-c1cccc(-c2cccc3c2C(C)(C)CCC3(C)C)c1 ; CC12CCCCC1(C)N(c1ccc3c(c1)c1ccccc1n3-c1cccc([Si](c3ccccc3)(c3ccccc3)c3cccc4ccccc34)c1)c1cnccc12 ; [2H]c1c(N2c3cnccc3C3(C)CCCCC23C)c([2H])c2c3ccccc3n(-c3ccccc3[Si](c3ccccc3)(c3ccc4ccccc4c3)c3ccc4ccccc4c3)c2c1[2H]]

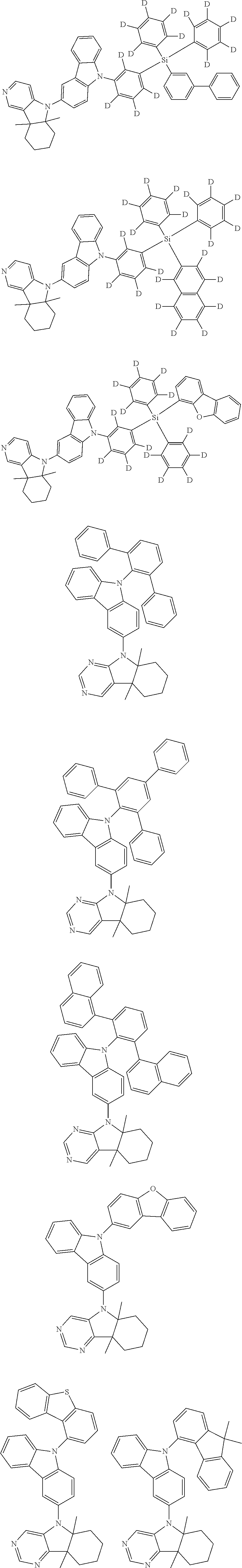

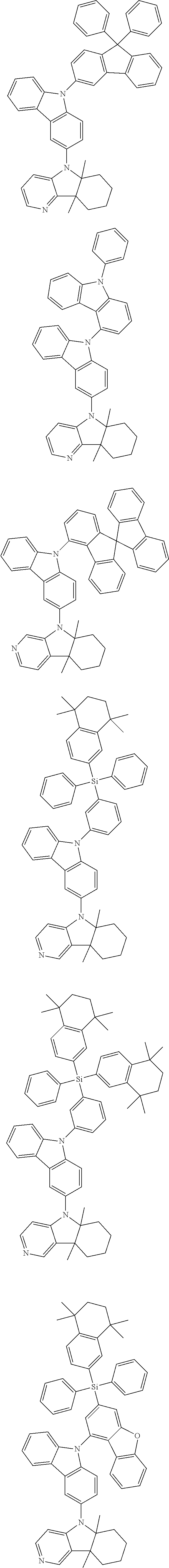

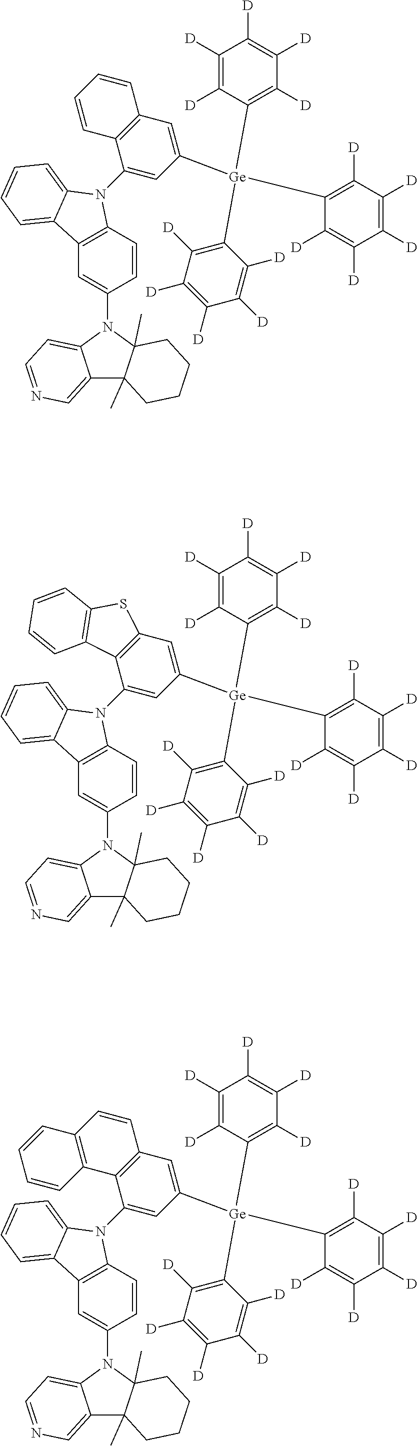

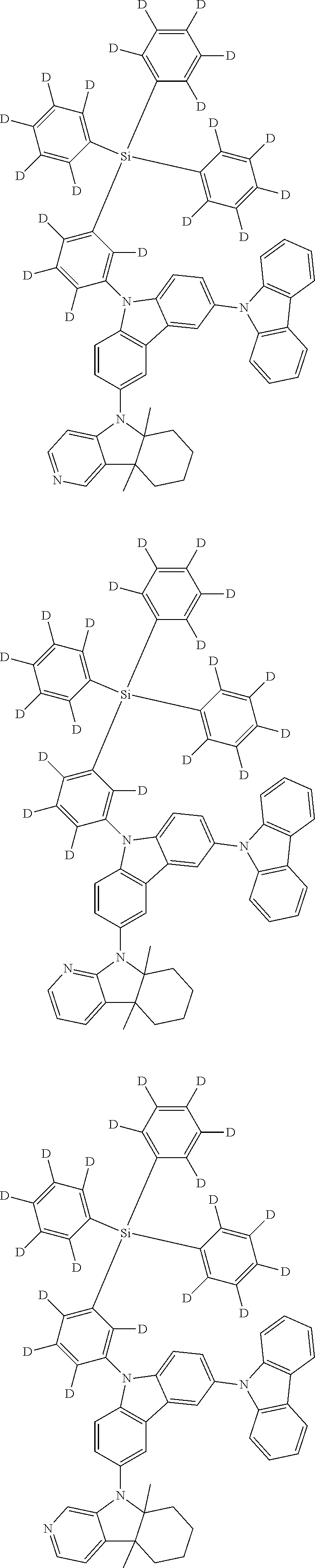

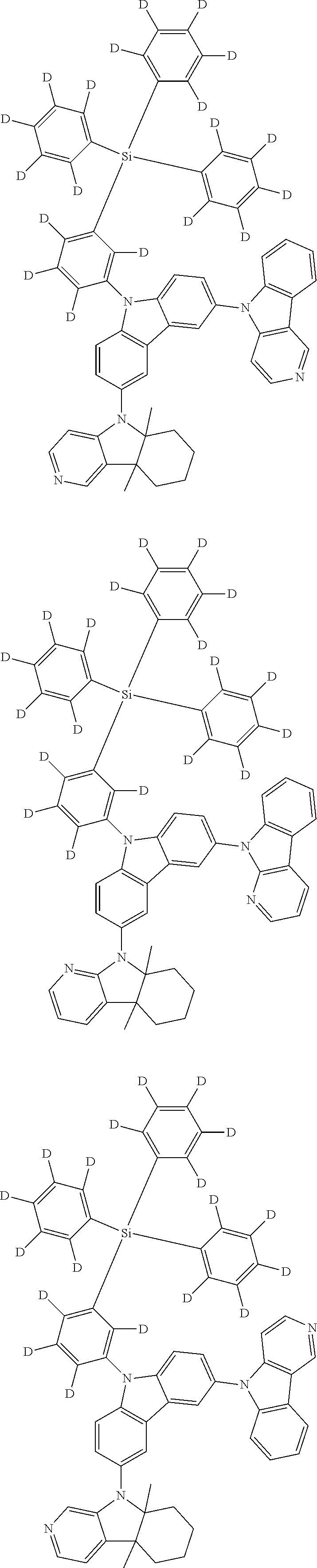

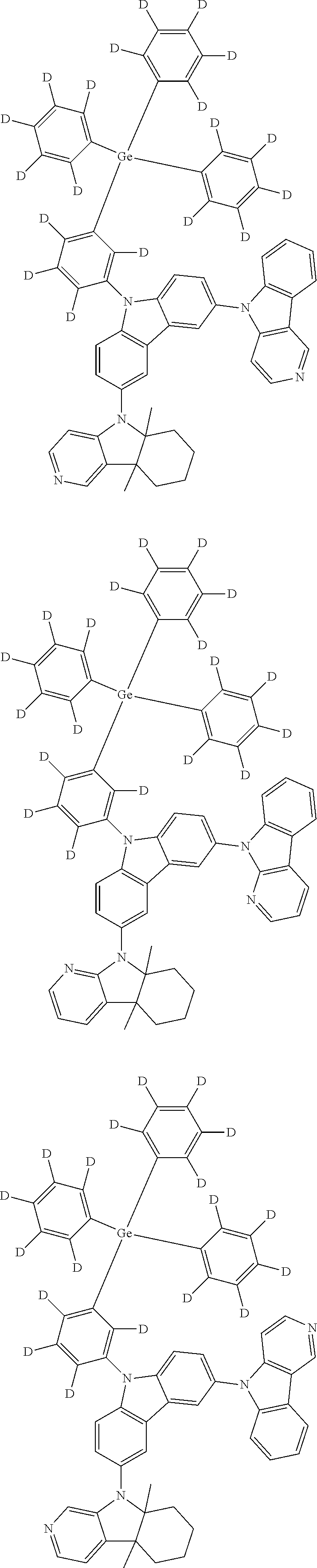

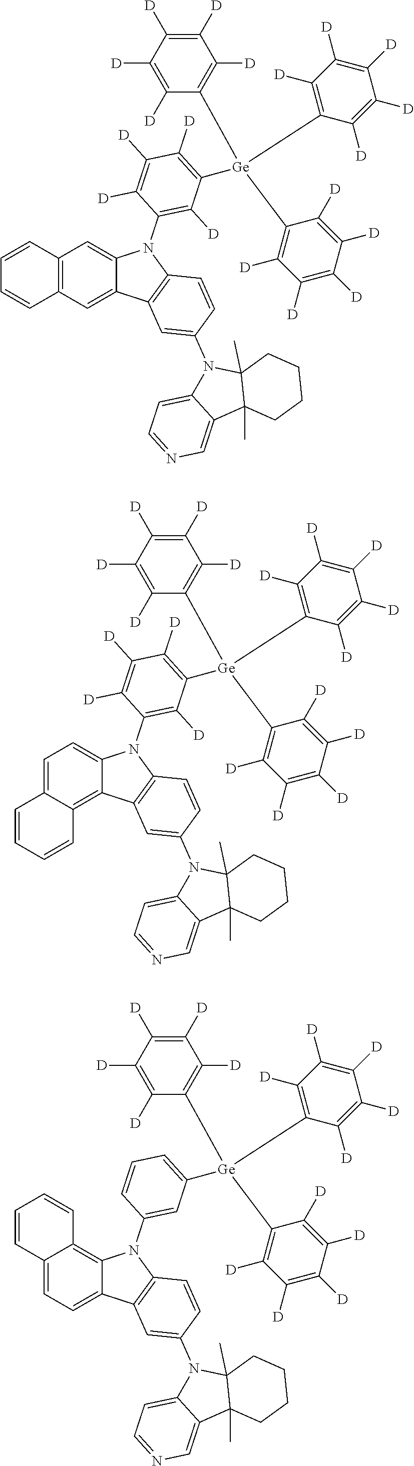

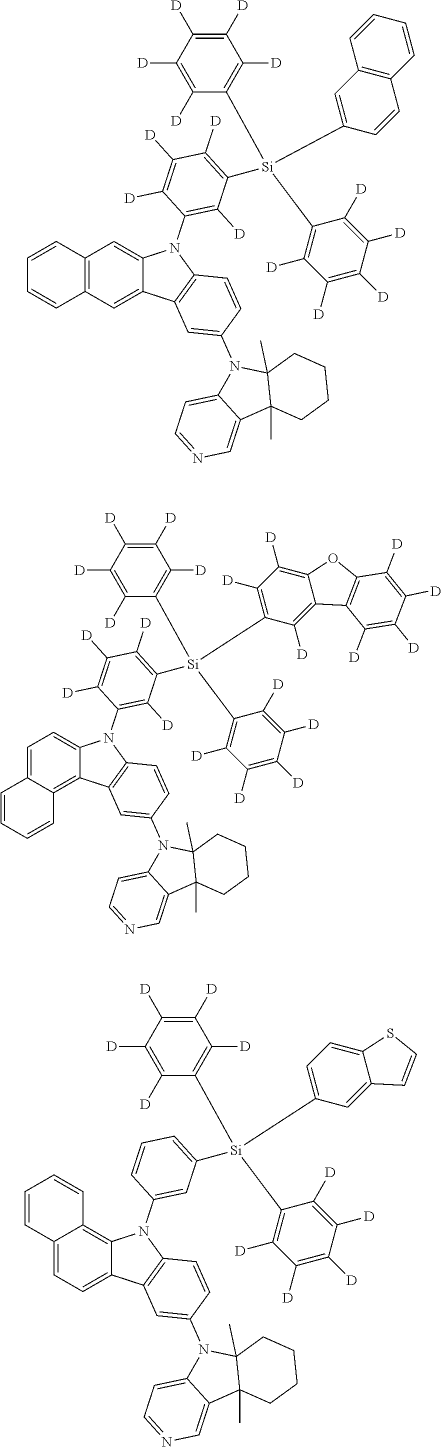

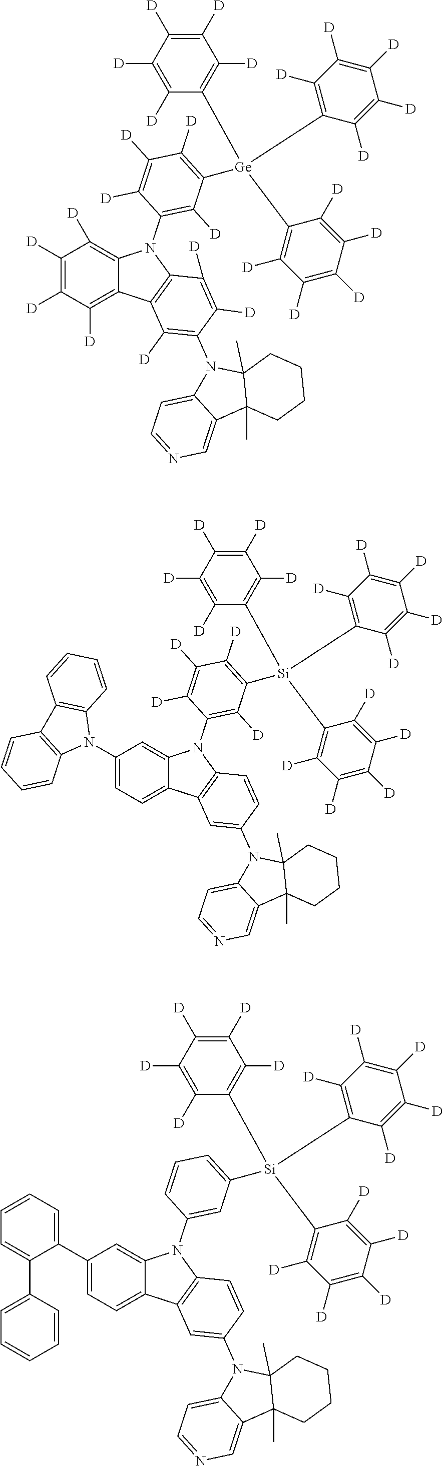

A more detailed description will be given concerning exemplary embodiments of the organic light emitting device according to the present invention.

The organic light emitting device of the present invention includes an anode, a hole transport layer, a light emitting layer, an electron transport layer, and a cathode. The organic light emitting device of the present invention may optionally further include a hole injecting layer between the anode and the hole transport layer and an electron injecting layer between the electron transport layer and the cathode. If necessary, the organic light emitting device of the present invention may further include one or two intermediate layers such as a hole blocking layer or an electron blocking layer. The organic light emitting device of the present invention may further include one or more organic layers such as a capping layer that have various functions depending on the desired characteristics of the device.

A specific structure of the organic light emitting device according to one embodiment of the present invention, a method for fabricating the device, and materials for the organic layers are as follows.

First, an anode material is coated on a substrate to form an anode. The substrate may be any of those used in general organic light emitting devices. The substrate is preferably an organic substrate or a transparent plastic substrate that is excellent in transparency, surface smoothness, ease of handling, and waterproofness. A highly transparent and conductive metal oxide such as indium tin oxide (ITO), indium zinc oxide (IZO), tin oxide (SnO₂) or zinc oxide (ZnO) is used as the anode material.

The hole injecting material is not specially limited so long as it is usually used in the art. Specific examples of such materials include 4,4′,4″-tris(2-naphthylphenyl-phenylamino)triphenylamine (2-TNATA), N,N′-di(1-naphthyl)-N,N′-diphenylbenzidine (NPD), N,N′-diphenyl-N,N′-bis(3-methylphenyl)-1,1′-biphenyl-4,4′-diamine (TPD), N,N′-diphenyl-N,N′-bis(4-(phenyl-m-tolylamino)phenyl) biphenyl-4,4′-diamine (DNTPD) and 1,4,5,8,9,11-hexaazatriphenylenehexacarbonitrile (HAT-CN).

The hole transport material is not specially limited so long as it is commonly used in the art. Examples of such materials include N,N′-bis(3-methylphenyl)-N,N′-diphenyl-(1,1-biphenyl)-4,4′-diamine (TPD) and N,N′-di(naphthalen-1-yl)-N,N′-diphenylbenzidine (α-NPD).

Subsequently, a hole auxiliary layer and a light emitting layer are sequentially stacked on the hole transport layer. A hole blocking layer may be optionally formed on the light emitting layer by vacuum thermal evaporation or spin coating. The hole blocking layer is formed as a thin film and blocks holes from entering a cathode through the organic light emitting layer. This role of the hole blocking layer prevents the lifetime and efficiency of the device from deteriorating. A material having a very low highest occupied molecular orbital (HOMO) energy level is used for the hole blocking layer. The hole blocking material is not particularly limited so long as it can transport electrons and has a higher ionization potential than the light emitting compound. Representative examples of suitable hole blocking materials include BAlq, BCP, and TPBI.

Examples of materials for the hole blocking layer include, but are not limited to, BAlq, BCP, Bphen, TPBI, TAZ, BeBq₂, OXD-7, and Liq.

An electron transport layer is deposited on the hole blocking layer by vacuum thermal evaporation or spin coating, and an electron injecting layer is formed thereon. A cathode metal is deposited on the electron injecting layer by vacuum thermal evaporation to form a cathode, completing the fabrication of the organic light emitting device.

For example, lithium (Li), magnesium (Mg), aluminum (Al), aluminum-lithium (Al—Li), calcium (Ca), magnesium-indium (Mg—In) or magnesium-silver (Mg—Ag) may be used as the metal for the formation of the cathode. The organic light emitting device may be of top emission type. In this case, a transmissive material such as ITO or IZO may be used to form the cathode.

A material for the electron transport layer functions to stably transport electrons injected from the cathode. The electron transport material may be any of those known in the art and examples thereof include, but are not limited to, quinoline derivatives, particularly tris(8-quinolinolato)aluminum (Alq₃), TAZ, BAlq, beryllium bis(benzoquinolin-10-olate) (Bebq₂), and oxadiazole derivatives such as PBD, BMD, and BND.

Each of the organic layers can be formed by a monomolecular deposition or solution process. According to the monomolecular deposition process, the material for each layer is evaporated into a thin film under heat and vacuum or reduced pressure. According to the solution process, the material for each layer is mixed with a suitable solvent and the mixture is then formed into a thin film by a suitable method such as ink-jet printing, roll-to-roll coating, screen printing, spray coating, dip coating or spin coating.

The organic light emitting device of the present invention can be used in a display or lighting system selected from flat panel displays, flexible displays, monochromatic flat panel lighting systems, white flat panel lighting systems, flexible monochromatic lighting systems, flexible white lighting systems, displays for automotives, displays for virtual reality, and displays for augmented reality.

The present invention will be more specifically explained with reference to the following synthesis examples and fabrication examples. However, these examples are provided to assist in understanding the invention and are not intended to limit the scope of the present invention.

### Synthesis Example 1: Synthesis of [HT-1]

### Synthesis Example 1-1: Synthesis of A-1

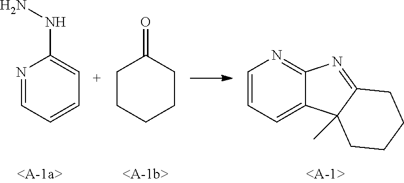

To a round bottom flask, <A-1a> (30 g), <A-1b> (30.9 g) and acetic acid (200 mL) were introduced, and then the mixture was stirred under reflux for 6 hours. After the reaction was completed, the reaction solution was basified with sodium hydroxide, and then neutralized by being extracted with water and ethylene acetate. After that, the organic layer was dried with magnesium sulfate. Then, the dried organic layer was concentrated under reduced pressure, and then separated and purified by column chromatography to obtain <A-1>. (27 g, 52.7%)

### Synthesis Example 1-2: Synthesis of A-2

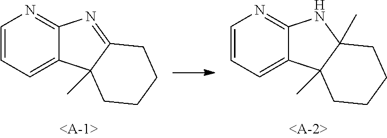

In a round bottom flask, <A-1> (50 g) was dissolved in toluene (150 mL), and then the temperature was lowered to 20° C. below zero. 1.6 M methyllithium (250 mL) was slowly added dropwise to the above solution, and then the mixture was reacted for 3 hours at 20° C. below zero. After the reaction was completed, a solution of toluene and water (1:1) (200 mL) was slowly poured into the reaction solution. The layers were separated, and the organic layer was dried with magnesium sulfate. The dried organic layer was concentrated under reduced pressure, and then separated and purified by column chromatography to obtain <A-2>. (44.7 g, 82.3%)

### Synthesis Example 1-3: Synthesis of A-3

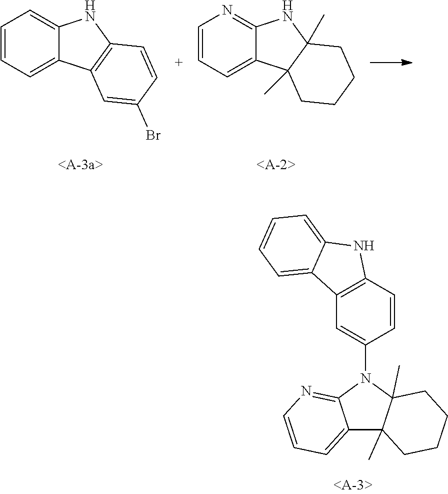

In a round bottom flask, <A-3a> (10 g), <A-2> (8.2 g), potassium acetate (12 g) and copper powder (0.26 g) were introduced to dimethylformamide (100 mL), and then the mixture was stirred under reflux for 9 hours at 120° C. When the reaction was finished, the result was washed with water, then suction filtered, and after washing the dimethylformamide with water, the result was extracted with dichloromethane. The organic layer was dried with MgSO₄, then concentrated under reduced pressure, and then separated and purified by column chromatography to obtain <A-3>. (12.2 g, 81.7%)

### Synthesis Example 1-4: Synthesis of A-4

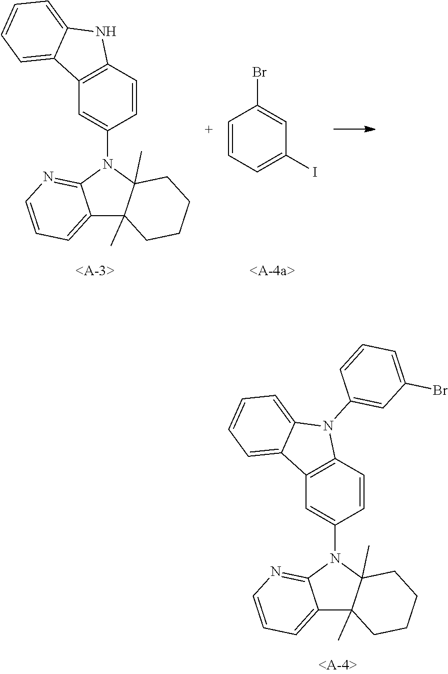 [SMILES: CC12CCCCC1(C)N(c1ccc3[nH]c4ccccc4c3c1)c1ncccc12 ; Brc1cccc(I)c1 ; CC12CCCCC1(C)N(c1ccc3c(c1)c1ccccc1n3-c1cccc(Br)c1)c1ncccc12]

To a round bottom flask, <A-3> (20 g), <A-4a> (15.4 g), tri-tert-butylphosphine (0.5 g) and sodium tert-butoxide (6.3 g) were introduced, and then the mixture was stirred under reflux for 18 hours at 100° C. After the reaction was finished, the result was extracted with water and dichloromethane, and then the organic layer was dried with MgSO₄. After that, the dried organic layer was concentrated under reduced pressure, and then separated and purified by column chromatography to obtain <A-4>. (25 g, 87.9%)

### Synthesis Example 1-5: Synthesis of A-5

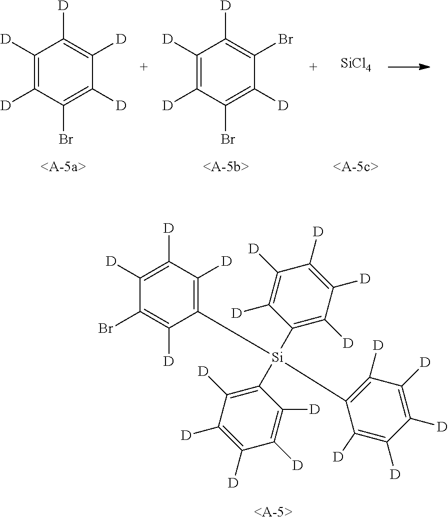

In a round bottom flask, <A-5a> (17.2 g) and diethyl ether (170 mL) were cooled to −78° C. under a nitrogen condition, and 1.6 M n-butyllithium (72.8 mL) was slowly added dropwise thereto. In another round bottom flask, <A-5b> (9.32 g) and diethyl ether (90 mL) were cooled to −78° C. under a nitrogen condition, and 1.6 M n-butyllithium (25.49 mL) was slowly added dropwise thereto. The mixtures in both flasks were stirred for 1 hour. The two reaction solutions were sequentially added dropwise to a round bottom flask containing a solution in which <A-5c> (6 g) was dissolved in diethyl ether (100 mL) cooed to −78° C., and then the mixture was stirred for 12 hours at room temperature. After the reaction was finished, water and ethyl acetate were introduced thereto to separate the layers, and the layers were dried with MgSO₄. The organic layer was concentrated under reduced pressure, and then separated and purified by column chromatography to obtain <A-5>. (24.4 g, 53%)

### Synthesis Example 1-6: Synthesis of A-6

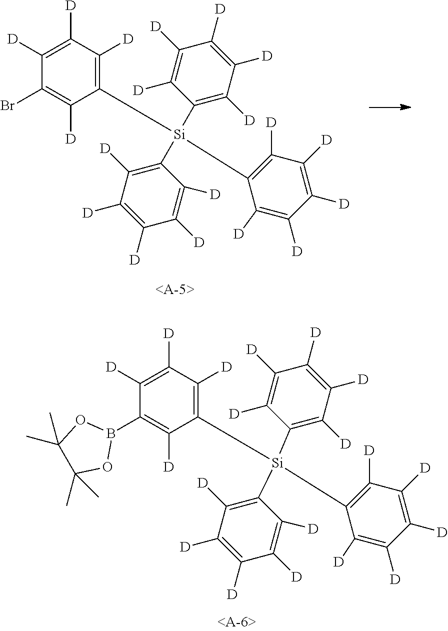 [SMILES: [2H]c1c([2H])c([2H])c([Si](c2c([2H])c([2H])c([2H])c([2H])c2[2H])(c2c([2H])c([2H])c([2H])c([2H])c2[2H])c2c([2H])c([2H])c([2H])c(Br)c2[2H])c([2H])c1[2H] ; [2H]c1c([2H])c([2H])c([Si](c2c([2H])c([2H])c([2H])c([2H])c2[2H])(c2c([2H])c([2H])c([2H])c([2H])c2[2H])c2c([2H])c([2H])c([2H])c(B3OC(C)(C)C(C)(C)O3)c2[2H])c([2H])c1[2H]]

To a round bottom flask, <A-5> (24.4 g), bis(pinacolato)diboron (18.5 g), [1,1′-bis(diphenylphosphino) ferrocene]dichloropalladium (II) (4.11 g), potassium acetate (16.53 g) and 1,4-dioxane (250 mL) were introduced under a nitrogen condition, and the mixture was refluxed for 12 hours. After the reaction was finished, the layers were separated, and dried with MgSO₄. The organic layer was concentrated under reduced pressure, and then separated and purified by column chromatography to obtain <A-6>. (20.6 g, 76%)

### Synthesis Example 1-7: Synthesis of [HT-1]

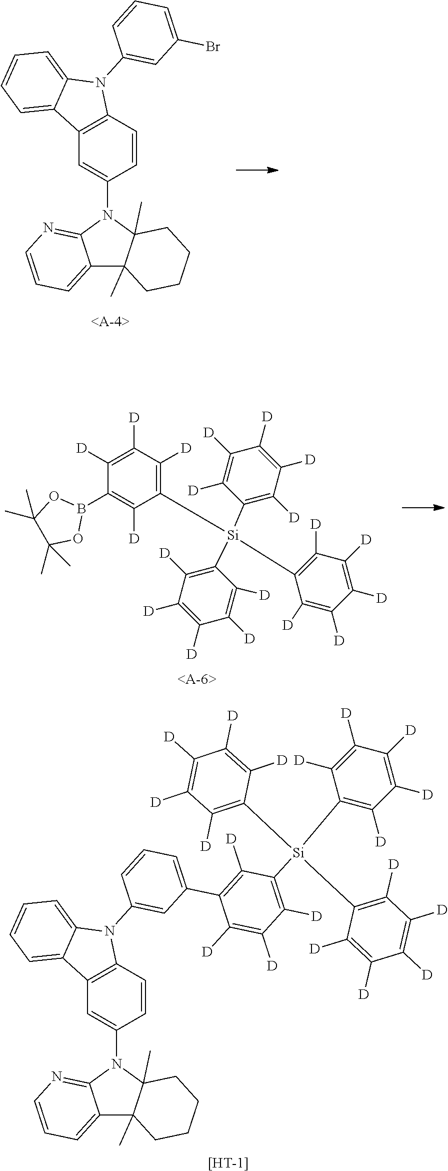

To a round bottom flask, <A-4> (18 g), <A-6> (19.9 g), tetrakis(triphenylphosphine) palladium (0) (2 g), potassium carbonate (11.9 g), 1,4-dioxane (180 mL) and water (40 mL) were introduced under a nitrogen condition, and the mixture was refluxed for 12 hours. After the reaction was finished, the layers were separated, and dried with MgSO₄. The organic layer was concentrated under reduced pressure, and then separated and purified by column chromatography to obtain [HT-1]. (16.8 g, 61%)

MS (MALDI-TOF): m/z 796.47 [M⁺]

### Synthesis Example 2: Synthesis of [HT-2]

### Synthesis Example 2-1: Synthesis of B-1

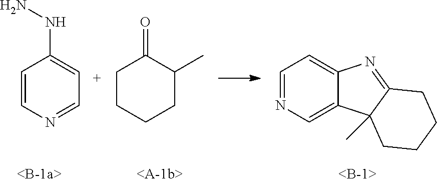

<B-1> was obtained in the same manner as in Synthesis Example 1-1, except that <B-1a> was used instead of <A-1a>. (Yield 51%)

### Synthesis Example 2-2: Synthesis of B-2

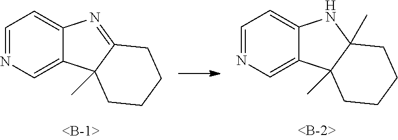

<B-2> was obtained in the same manner as in Synthesis Example 1-2, except that <B-1> was used instead of <A-1>. (Yield 81%)

### Synthesis Example 2-3: Synthesis of B-3

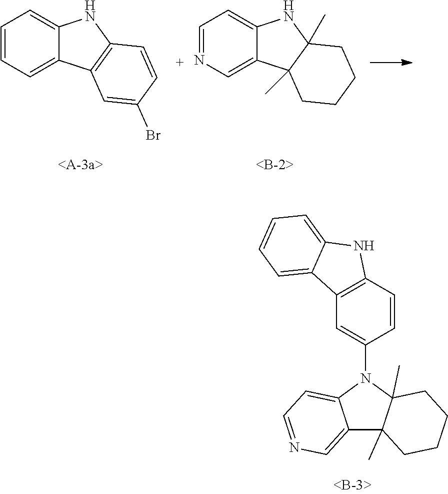

<B-3> was obtained in the same manner as in Synthesis Example 1-3, except that <B-2> was used instead of <A-2>. (Yield 77.4%)

### Synthesis Example 2-4: Synthesis of [HT-2]

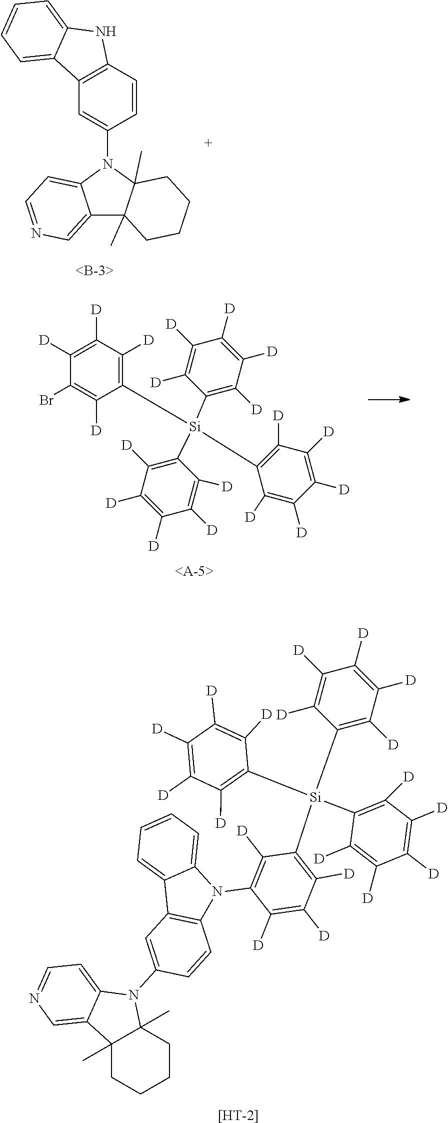

[HT-2] was obtained in the same manner as in Synthesis Example 1-3, except that <A-5> was used instead of <A-3a>, and <B-3> was used instead of <A-2>. (Yield 77.6%)

MS (MALDI-TOF): m/z 720.44 [M⁺]

### Synthesis Example 3: Synthesis of [HT-3]

### Synthesis Example 3-1: Synthesis of C-1

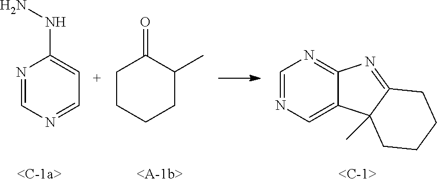

<C-1> was obtained in the same manner as in Synthesis Example 1-1, except that <C-1a> was used instead of <A-1a>. (Yield 45%)

### Synthesis Example 3-2: Synthesis of C-2

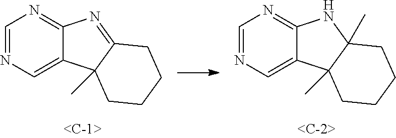

<C-2> was obtained in the same manner as in Synthesis Example 1-2, except that <C-1> was used instead of <A-1>. (Yield 82%)

### Synthesis Example 3-3: Synthesis of C-3

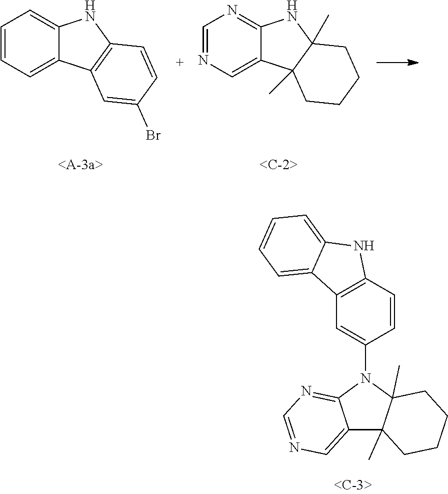 [SMILES: Brc1ccc2[nH]c3ccccc3c2c1 ; CC12CCCCC1(C)c1cncnc1N2 ; CC12CCCCC1(C)N(c1ccc3[nH]c4ccccc4c3c1)c1ncncc12]

<C-3> was obtained in the same manner as in Synthesis Example 1-3, except that <C-2> was used instead of <A-2>. (Yield 79%)

### Synthesis Example 3-4: Synthesis of [HT-3]

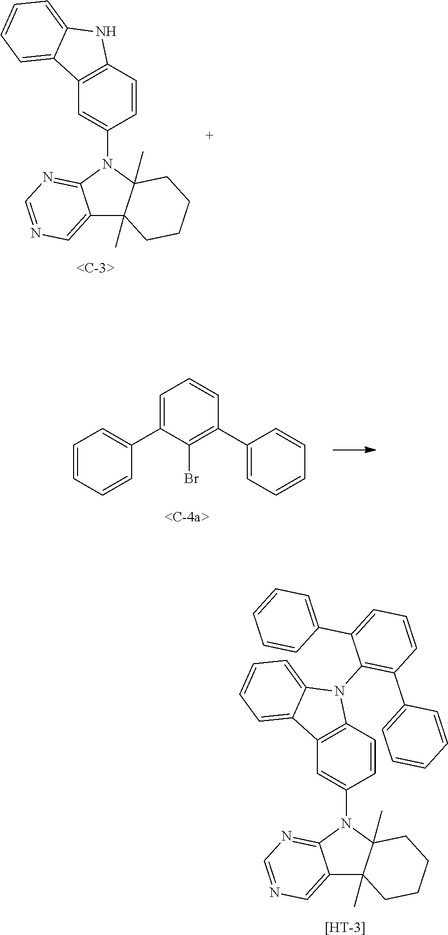

[HT-3] was obtained in the same manner as in Synthesis Example 1-3, except that <C-4a> was used instead of <A-3a>, and <C-3> was used instead of <A-2>. (Yield 76%)

MS (MALDI-TOF): m/z 596.29 [M⁺]

### Synthesis Example 4: Synthesis of [HT-4]

### Synthesis Example 4-1: Synthesis of D-1

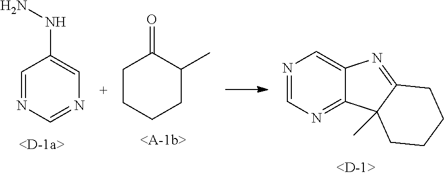

<D-1> was obtained in the same manner as in Synthesis Example 1-1, except that <D-1a> was used instead of <A-1a>. (Yield 49%)

### Synthesis Example 4-2: Synthesis of D-2

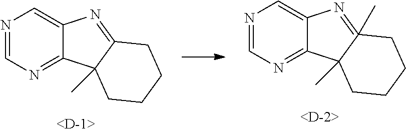

<D-2> was obtained in the same manner as in Synthesis Example 1-2, except that <D-1> was used instead of <A-1>. (Yield 80%)

### Synthesis Example 4-3: Synthesis of D-3

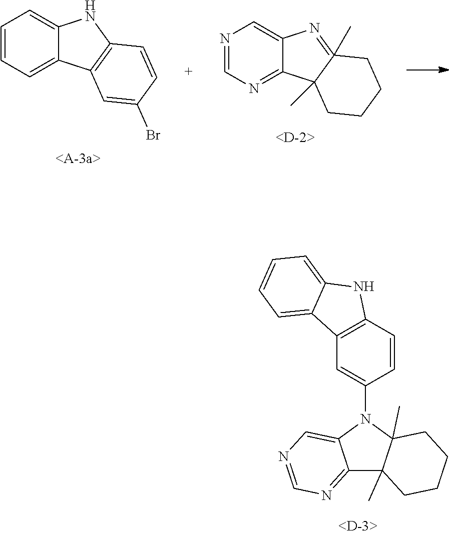

<D-3> was obtained in the same manner as in Synthesis Example 1-3, except that <D-2> was used instead of <A-2>. (Yield 78%)

### Synthesis Example 4-4: Synthesis of [HT-4]

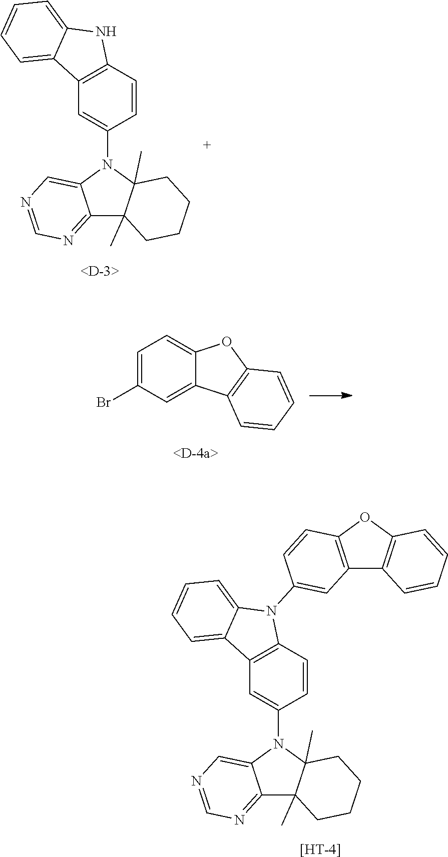

[HT-4] was obtained in the same manner as in Synthesis Example 1-3, except that <D-4a> was used instead of <A-3a>, and <D-3> was used instead of <A-2>. (Yield 83%)

MS (MALDI-TOF): m/z 534.24 [M⁺]

### Synthesis Example 5: Synthesis of [HT-5]

### Synthesis Example 5-1: Synthesis of E-1

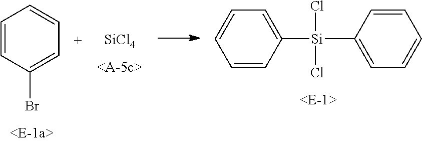

<E-1> was obtained in the same manner as in Synthesis Example 1-5, except that <E-1a> was used instead of <A-5a> and <A-5b>. (Yield 62%)

### Synthesis Example 5-2: Synthesis of E-2

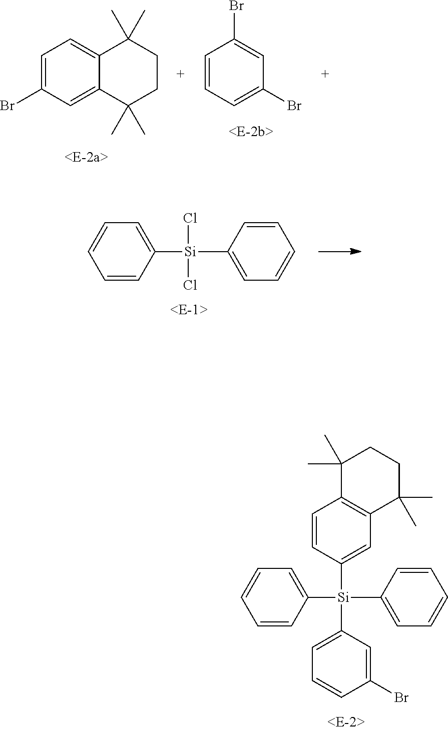 [SMILES: CC1(C)CCC(C)(C)c2cc(Br)ccc21 ; Brc1cccc(Br)c1 ; Cl[Si](Cl)(c1ccccc1)c1ccccc1 ; CC1(C)CCC(C)(C)c2cc([Si](c3ccccc3)(c3ccccc3)c3cccc(Br)c3)ccc21]

<E-2> was obtained in the same manner as in Synthesis Example 1-5, except that <E-2a> was used instead of <A-5a>, <E-2b> was used instead of <A-5b>, and <E-1> was used instead of <A-5c>. (Yield 57%)

### Synthesis Example 5-3: Synthesis of [HT-5]

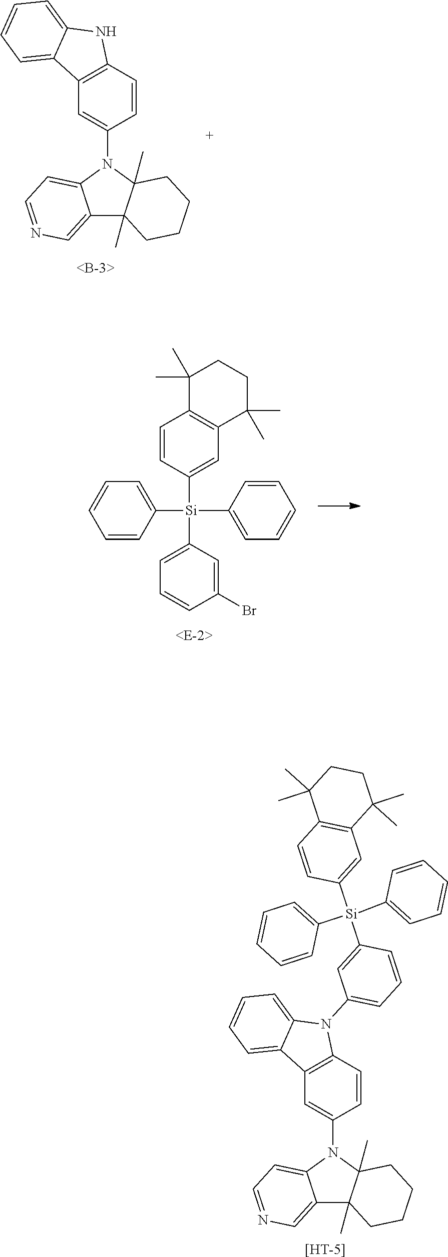

[HT-5] was obtained in the same manner as in Synthesis Example 1-3, except that <E-2> was used instead of <A-3a>, and <B-3> was used instead of <A-2>. (Yield 76.2%)

MS (MALDI-TOF): m/z 811.43 [M⁺]

### Synthesis Example 6: Synthesis of [HT-6]

### Synthesis Example 6-1: Synthesis of F-1

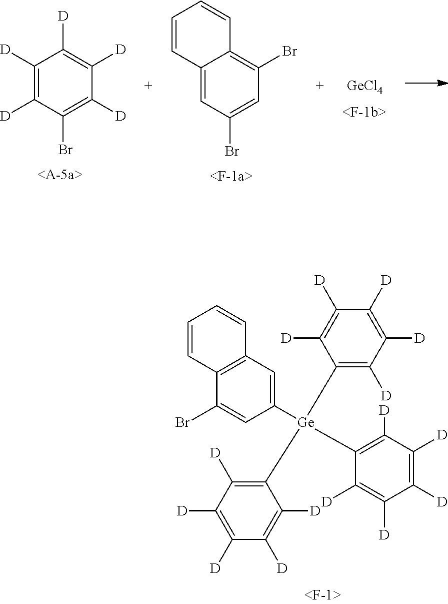

<F-1> was obtained in the same manner as in Synthesis Example 1-5, except that <F-1a> was used instead of <A-5b>, and <F-1b> was used instead of <A-5c>. (Yield 54%)

### Synthesis Example 6-2: Synthesis of [HT-6]

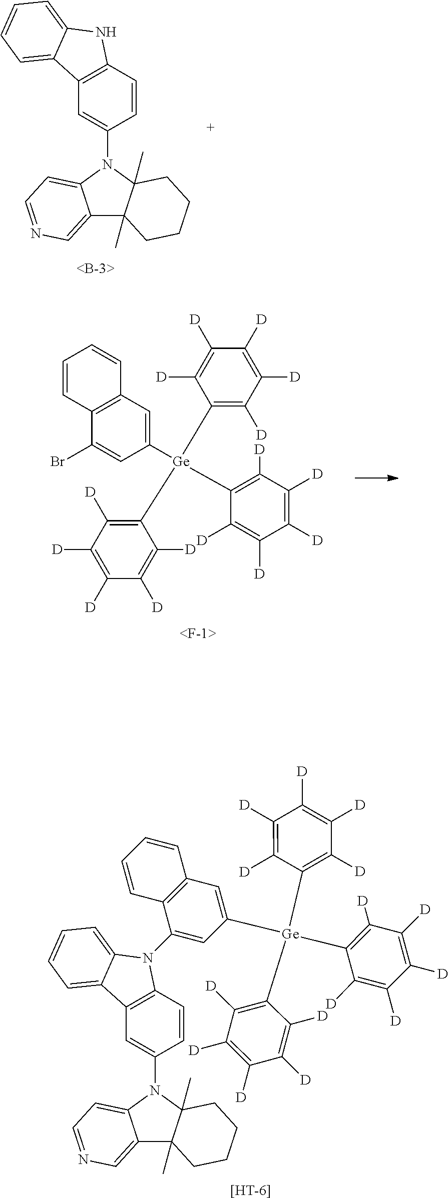

[HT-6] was obtained in the same manner as in Synthesis Example 1-3, except that <F-1> was used instead of <A-3a>, and <B-3> was used instead of <A-2>. (Yield 67%)

MS (MALDI-TOF): m/z 812.38 [M⁺]

### Synthesis Example 7: Synthesis of [HT-7]

### Synthesis Example 7-1: Synthesis of G-1

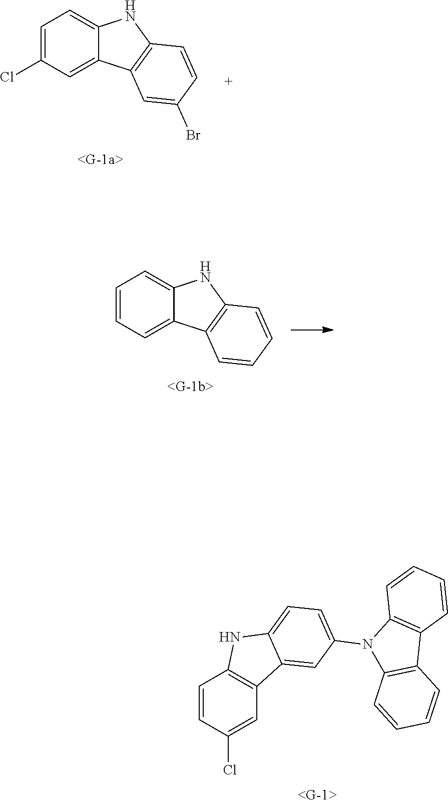 [SMILES: Clc1ccc2[nH]c3ccc(Br)cc3c2c1 ; c1ccc2c(c1)[nH]c1ccccc12 ; Clc1ccc2[nH]c3ccc(-n4c5ccccc5c5ccccc54)cc3c2c1]

<G-1> was obtained in the same manner as in Synthesis Example 1-3, except that <G-1a> was used instead of <A-3a>, and <G-1b> was used instead of <A-2>. (Yield 77.4%)

### Synthesis Example 7-2: Synthesis of G-2

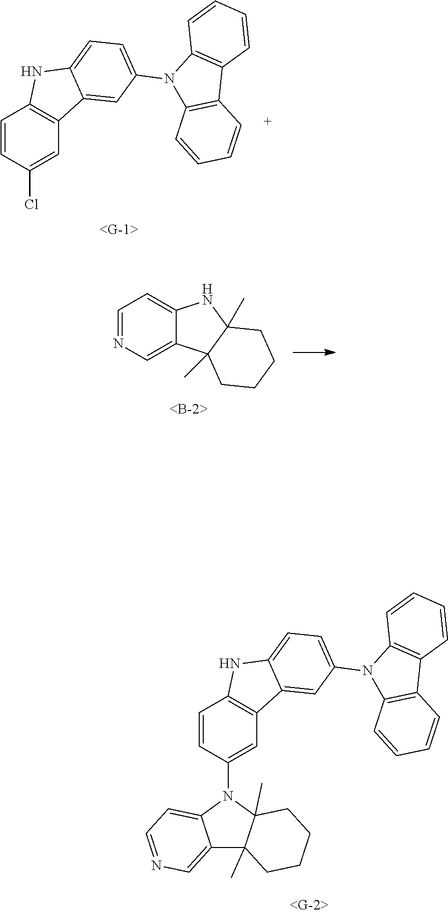 [SMILES: Clc1ccc2[nH]c3ccc(-n4c5ccccc5c5ccccc54)cc3c2c1 ; CC12CCCCC1(C)c1cnccc1N2 ; CC12CCCCC1(C)N(c1ccc3[nH]c4ccc(-n5c6ccccc6c6ccccc65)cc4c3c1)c1ccncc12]

<G-2> was obtained in the same manner as in Synthesis Example 1-3, except that <G-1> was used instead of <A-3a>, and <B-2> was used instead of <A-2>. (Yield 75.2%)

### Synthesis Example 7-3: Synthesis of [HT-7]

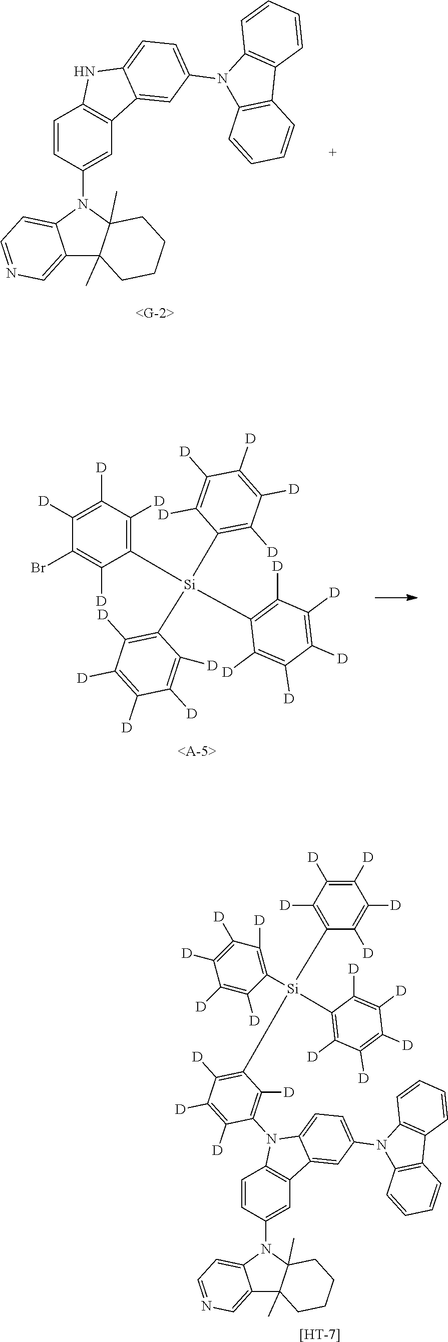

[HT-7] was obtained in the same manner as in Synthesis Example 1-3, except that <A-5> was used instead of <A-3a>, and <G-2> was used instead of <A-2>. (Yield 73.2%)

MS (MALDI-TOF): m/z 885.50 [M⁺]

### Synthesis Example 8: Synthesis of [HT-8]

### Synthesis Example 8-1: Synthesis of H-1

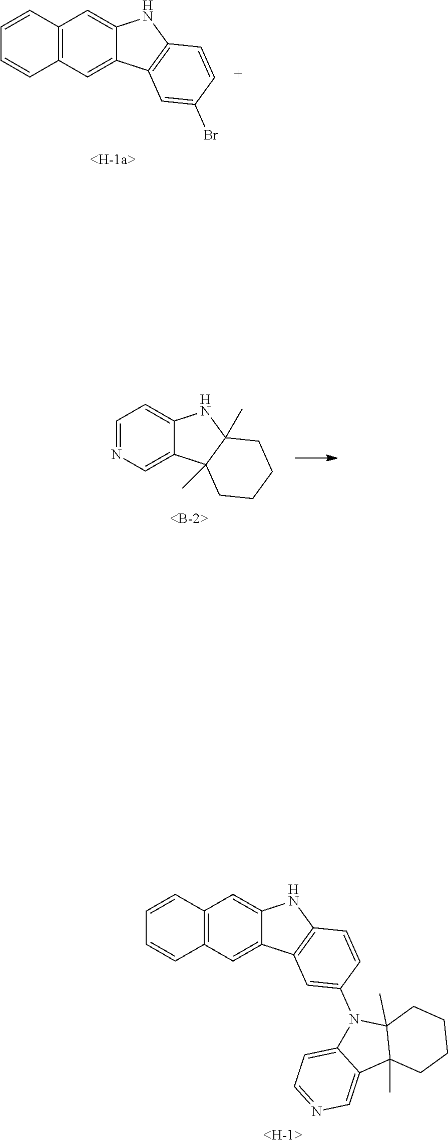 [SMILES: Brc1ccc2[nH]c3cc4ccccc4cc3c2c1 ; CC12CCCCC1(C)c1cnccc1N2 ; CC12CCCCC1(C)N(c1ccc3[nH]c4cc5ccccc5cc4c3c1)c1ccncc12]

<H-1> was obtained in the same manner as in Synthesis Example 1-3, except that <H-1a> was used instead of <A-3a>, and <B-2> was used instead of <A-2>. (Yield 78.1%)

### Synthesis Example 8-2: Synthesis of [HT-8]

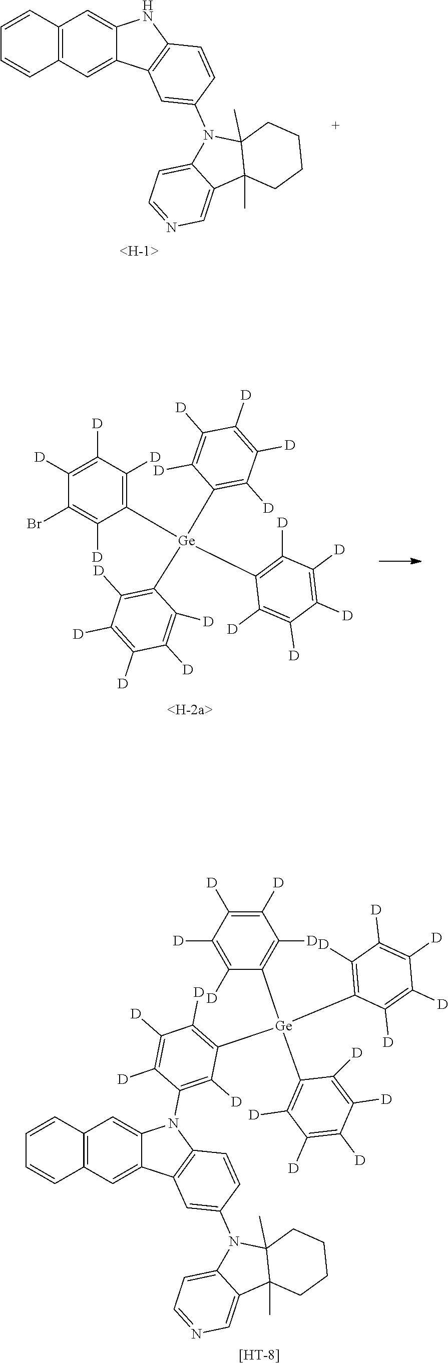

[HT-8] was obtained in the same manner as in Synthesis Example 1-3, except that <H-2a> was used instead of <A-3a>, and <H-1> was used instead of <A-2>. (Yield 74.2%)

MS (MALDI-TOF): m/z 816.40 [M⁺]

### Examples 1 to 8: Manufacture of Organic Light Emitting Device

An ITO glass was patterned to have a light emitting area of 2 mm×2 mm, and then cleaned. The ITO glass was installed in a vacuum chamber, and after setting the base pressure at 1×10⁻⁶ torr, HAT-CN (50 Å) was deposited as a hole injecting layer, BCFN (600 Å) was deposited as a hole transport layer, and then PBCz (50 Å) was deposited as an electron blocking layer on the ITO glass. As a light emitting layer, the first host compound and the second host compound according to the present invention, and the following PBD as a dopant compound were mixed in 12 wt % of the total weight of the light emitting layer and then deposited (350 Å). Then, mSiTrz (50 Å) was deposited as a hole blocking layer, mSiTrz: Liq (300 Å) in a ratio of 1:1 were deposited as an electron injecting and transport layer, and then Liq (10 Å) was deposited as an electron injecting layer sequentially, and Al (1,000 Å) was deposited as a cathode to manufacture an organic light emitting device. Luminous characteristics of the organic light emitting device were measured at 0.4 mA.

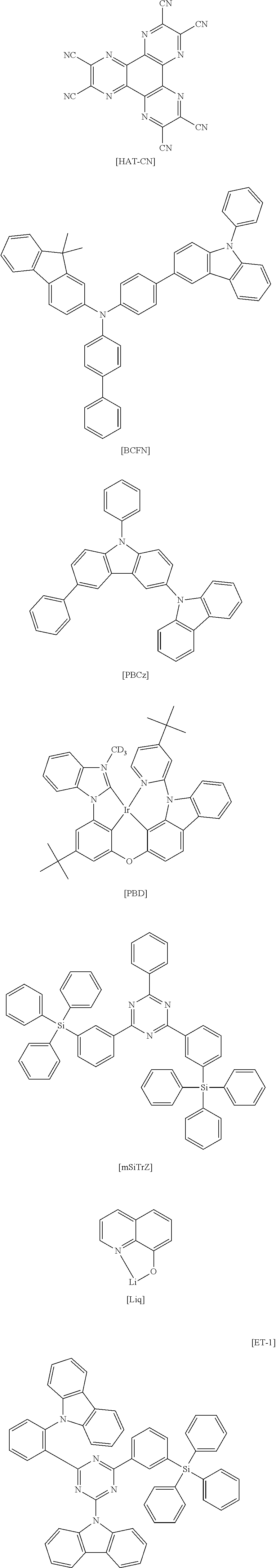

### Comparative Examples 1 and 2

Organic light emitting devices for Comparative Examples were manufactured in the same manner as in Examples, except that the following [RH-1] and [RH-2] were used as the host compound instead of the compound according to the present invention in the device structures of Examples, and luminous characteristics of the organic light emitting devices were measured at 0.4 mA. The structures of [RH-1] and [RH-2] are as follows.

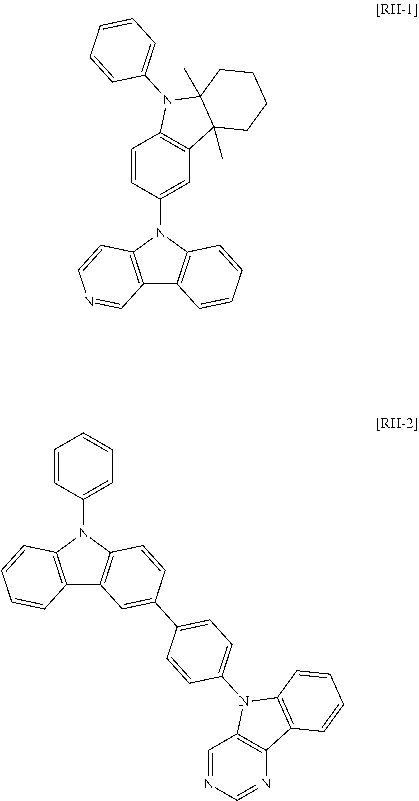 [SMILES: CC12CCCCC1(C)N(c1ccccc1)c1ccc(-n3c4ccccc4c4cnccc43)cc12 ; c1ccc(-n2c3ccccc3c3cc(-c4ccc(-n5c6ccccc6c6ncncc65)cc4)ccc32)cc1]

TABLE 1

| | | | | External | | |
|---|---|---|---|---|---|---|
| | | | Driving | Quantum | | Light |
| | | | Voltage | Efficiency | Lifetime | Emitting |
| Category | Host (1:1) | | (V) | (EQE, %) | (T95, hr) | Color |
| Example 1 | HT-1 | ET-1 | 4.4 | 16.1 | 153 | Blue |
| Example 2 | HT-2 | ET-1 | 4.3 | 16.7 | 162 | Blue |
| Example 3 | HT-3 | ET-1 | 4.3 | 14.3 | 125 | Blue |
| Example 4 | HT-4 | ET-1 | 4.3 | 14.6 | 120 | Blue |
| Example 5 | HT-5 | ET-1 | 4.3 | 16.4 | 157 | Blue |
| Example 6 | HT-6 | ET-1 | 4.4 | 15.8 | 148 | Blue |
| Example 7 | HT-7 | ET-1 | 4.4 | 15.1 | 142 | Blue |
| Example 8 | HT-8 | ET-1 | 4.4 | 15.4 | 135 | Blue |
| Comparative | RH-1 | ET-1 | 4.7 | 12.1 | 90 | Blue |
| Example 1 | | | | | | |
| Comparative | RH-2 | ET-1 | 4.8 | 10.3 | 82 | Blue |
| Example 2 | | | | | | |

As shown in [Table 1], the device employing the compound according to the present invention as a light emitting layer host compound in the organic light emitting device may be embodied as a high-efficiency and long-lifetime organic light emitting device with excellent external quantum efficiency and lifetime properties at a low driving voltage compared to the devices (Comparative Examples 1 and 2) employing compounds widely used in the related art having structures in contrast to the characteristic structures of the compound according to the present invention.

### Examples 9 to 16: Manufacture of Organic Light Emitting Device

Organic light emitting devices were manufactured in the same manner as in Examples 1 to 8, except that, as the dopant compound according to the present invention, a compound of the following BD-1 compound was further mixed and used in 0.5 wt % of the total weight of the light emitting layer. Luminous characteristics of the organic light emitting devices were measured at 0.4 mA.

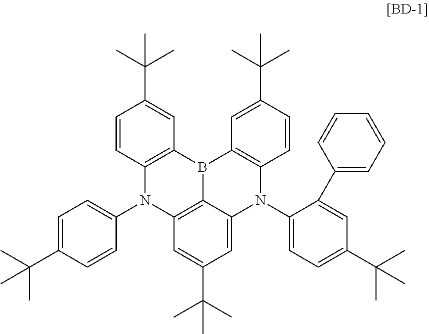 [SMILES: CC(C)(C)c1ccc(N2c3ccc(C(C)(C)C)cc3B3c4cc(C(C)(C)C)ccc4N(c4ccc(C(C)(C)C)cc4-c4ccccc4)c4cc(C(C)(C)C)cc2c43)cc1]

### Comparative Examples 3 and 4

Organic light emitting devices for Comparative Examples were manufactured in the same manner as in Examples, except that [RH-1] and [RH-2] were used as the host compound instead of the compound according to the present invention in the device structures of Examples. Luminous characteristics of the organic light emitting devices were measured at 0.4 mA.

TABLE 2

| | | | | External | | |
|---|---|---|---|---|---|---|
| | | | Driving | Quantum | | Light |
| | | | Voltage | Efficiency | Lifetime | Emitting |
| Category | Host (1:1) | | (V) | (EQE, %) | (T95, hr) | Color |
| Example 9 | HT-1 | ET-1 | 4.5 | 17.1 | 170 | Blue |
| Example 10 | HT-2 | ET-1 | 4.4 | 17.7 | 185 | Blue |
| Example 11 | HT-3 | ET-1 | 4.4 | 15.3 | 150 | Blue |
| Example 12 | HT-4 | ET-1 | 4.5 | 15.6 | 147 | Blue |
| Example 13 | HT-5 | ET-1 | 4.4 | 17.4 | 174 | Blue |
| Example 14 | HT-6 | ET-1 | 4.4 | 16.8 | 173 | Blue |
| Example 15 | HT-7 | ET-1 | 4.5 | 16.1 | 160 | Blue |
| Example 16 | HT-8 | ET-1 | 4.4 | 16.4 | 153 | Blue |
| Comparative | RH-1 | ET-1 | 4.8 | 12.5 | 103 | Blue |
| Example 3 | | | | | | |
| Comparative | RH-2 | ET-1 | 4.8 | 10.8 | 95 | Blue |
| Example 4 | | | | | | |

As shown in [Table 2], the device employing the compound according to the present invention as a light emitting layer host compound in the organic light emitting device may be embodied as a high-efficiency and long-lifetime organic light emitting device with excellent external quantum efficiency and lifetime properties at a low driving voltage compared to the devices (Comparative Examples 3 and 4) employing compounds widely used in the related art having structures in contrast to the characteristic structures of the compound according to the present invention.

### Examples 17 to 21: Manufacture of Organic Light Emitting Device

Organic light emitting devices were manufactured and experimented in the same manner as in Examples 1 to 8, except that the host compound according to the present invention was used alone as the host in the light emitting layer. Luminous characteristics of the organic light emitting devices were measured at 0.4 mA.

### Comparative Examples 5 and 6

Organic light emitting devices for Comparative Examples were manufactured in the same manner as in Examples, except that [RH-1] and [RH-2] were used as the host compound instead of the compound according to the present invention in the device structures of Examples. Luminous characteristics of the organic light emitting devices were measured at 0.4 mA.

TABLE 3

| | | | External | | |
|---|---|---|---|---|---|
| | | Driving | Quantum | | Light |
| | Host | Voltage | Efficiency | Lifetime | Emitting |
| Category | (1:1) | (V) | (EQE, %) | (T95, hr) | Color |
| Example 17 | HT-1 | 5.3 | 14.3 | 133 | Blue |
| Example 18 | HT-2 | 5.2 | 15.3 | 142 | Blue |
| Example 19 | HT-3 | 5.3 | 12.5 | 105 | Blue |
| Example 20 | HT-4 | 5.3 | 12.8 | 100 | Blue |
| Example 21 | HT-5 | 5.2 | 14.4 | 137 | Blue |
| Example 22 | HT-6 | 5.2 | 13.9 | 128 | Blue |
| Example 23 | HT-7 | 5.3 | 13.4 | 122 | Blue |
| Example 24 | HT-8 | 5.3 | 12.9 | 115 | Blue |
| Comparative | RH-1 | 5.5 | 9.8 | 72 | Blue |
| Example 7 | | | | | |
| Comparative | RH-2 | 5.6 | 8.3 | 54 | Blue |
| Example 8 | | | | | |

As shown in [Table 3], the device employing the host compound according to the present invention as a light emitting layer host compound in the organic light emitting device may be embodied as a high-efficiency and long-lifetime organic light emitting device with excellent external quantum efficiency and lifetime properties at a low driving voltage compared to the devices (Comparative Examples 5 and 6) employing compounds widely used in the related art having structures in contrast to the characteristic structures of the compound according to the present invention.

1. An organic light emitting device comprising:
a first electrode;
a second electrode opposite to the first electrode; and
an organic layer interposed between the first electrode and the second electrode,
wherein the organic layer includes a light emitting layer including a host and a dopant; and
the host includes at least one type of organic compound represented by the following [Formula 1]: 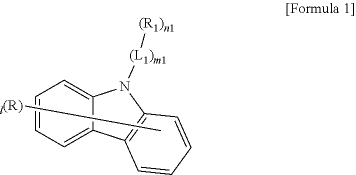
in [Formula 1],
L₁s are the same as or different from each other, and each independently a single bond, or any one selected from substituted or unsubstituted C₆-C₃₀ arylene, substituted or unsubstituted C₂-C₃₀ heteroarylene, and substituted or unsubstituted divalent cyclic groups in which a C₃-C₂₄ aliphatic ring and a C₅-C₂₄ aromatic ring are fused together;
R and R₁ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted cyclic groups in which a C₃-C₃₀ aliphatic ring and a C₅-C₃₀ aromatic ring are fused together, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano and halogen;
however, at least one of Rs of [Formula 1] is [Structural Formula 1], 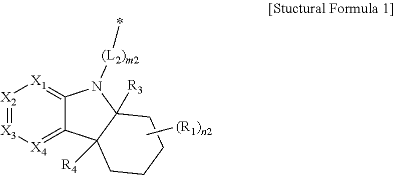
in [Structural Formula 1],
‘*’ means a bonding site linked to a carbon atom (C) in the carbazole of [Formula 1];
X₁ to X₄ are the same as or different from each other and each independently N or CR₅, and at least one of X₁ to X₄ is N;
L₂s are the same as or different from each other, and each independently a single bond, or any one selected from substituted or unsubstituted C₆-C₃₀ arylene, substituted or unsubstituted C₂-C₃₀ heteroarylene, and substituted or unsubstituted divalent cyclic groups in which a C₃-C₂₄ aliphatic ring and a C₅-C₂₄ aromatic ring are fused together;
R₂ and R₅ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted cyclic groups in which a C₃-C₃₀ aliphatic ring and a C₅-C₃₀ aromatic ring are fused together, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano and halogen;
R₃ and R₄ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, and substituted or unsubstituted cyclic groups in which a C₃-C₃₀ aliphatic ring and a C₅-C₃₀ aromatic ring are fused together;
n₁ is an integer of 1 to 3, and when it is an integer of 2 or greater, R₁s are the same as or different from each other;
n₂ is an integer of 4, and R₂s are the same as or different from each other;
m₁ and m₂ are each independently an integer of 1 to 3, and when they are integers of 2 or greater, L₁s and L₂s are the same as or different from each other;
l is an integer of 8, and Rs except for the part linked to [Structural Formula 1] are the same as or different from each other;
the plurality of adjacent Rs and R₅s are optionally linked to each other to further form an alicyclic or aromatic monocyclic or polycyclic ring; and
the term ‘substituted’ in the ‘substituted or unsubstituted’ in [Formula 1] and [Structural Formula 1] means being substituted with one or more substituents selected from the group consisting of deuterium, tritium, C₁-C₂₄ alkyl, C₁-C₂₄ haloalkyl, C₂-C₂₄ alkenyl, C₂-C₂₄ alkynyl, C₃-C₃₀ cycloalkyl, C₁-C₂₄ heteroalkyl, C₆-C₃₀ aryl, C₇-C₃₀ arylalkyl, C₇-C₃₀ alkylaryl, C₂-C₃₀ heteroaryl, C₂-C₃₀ heteroarylalkyl, cyclic groups in which a C₃-C₂₄ aliphatic ring and a C₅-C₂₄ aromatic ring are fused together, C₁-C₂₄ alkoxy, C₁-C₃₀ amine, C₁-C₃₀ silyl, C₁-C₃₀ germanium, C₆-C₂₄ aryloxy, C₆-C₂₄ arylthionyl, cyano, halogen, hydroxyl and nitro, and when there are two or more substituents, they are the same as or different from each other, and one or more hydrogen atoms in each of the substituents are optionally substituted with deuterium or tritium atoms.

2. The organic light emitting device according to claim 1, wherein at least one of R₁s in [Formula 1] is any one selected from substituted or unsubstituted C₁-C₄₀ silyl and substituted or unsubstituted C₁-C₄₀ germanium.

3. The organic light emitting device according to claim 1, wherein the rest except for the case in which any one of Rs in [Formula 1] is [Structural Formula 1] is hydrogen or deuterium.

4. The organic light emitting device according to claim 1, wherein m₁ in [Formula 1] is an integer of 1, and at the same time, L₁ is substituted or unsubstituted C₆-C₃₀ arylene.

5. The organic light emitting device according to claim 1, wherein R₂ to R₄ in [Structural Formula 1] are the same as or different from each other, and each independently any one selected from hydrogen, deuterium and substituted or unsubstituted C₁-C₁₀ alkyl.

6. The organic light emitting device according to claim 1, wherein R₅ of CR₅ except for the case in which X₁ to X₄ in [Structural Formula 1] are N is hydrogen or deuterium.

7. The organic light emitting device according to claim 1, wherein m₂ in [Structural Formula 1] is an integer of 1, and at the same time, L₂ is a single bond.

8. The organic light emitting device according to claim 1, wherein at least one deuterium atom substitutes in [Formula 1].

9. The organic light emitting device according to claim 1, wherein, in addition to the organic compound represented by [Formula 1], one or more types of host compounds different therefrom are mixed or stacked and used as the host in the light emitting layer of the organic light emitting device.

10. The organic light emitting device according to claim 1, wherein the dopant in the light emitting layer of the organic light emitting device includes at least one organometallic compound including a transition metal.

11. The organic light emitting device according to claim 10, wherein, as the dopant in the light emitting layer of the organic light emitting device, one or more types of polycyclic compounds represented by the following [Formula 2] are mixed and used in addition to the organometallic compound including a transition metal, or a polycyclic compound represented by the following [Formula 2] is stacked and used above or below the layer including the organometallic compound: 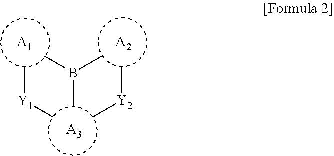
in [Formula 2],
Y₁ and Y₂ are the same as or different from each other, and each independently any one selected from O, S, NR₁₁, CR₁₂R₁₃, SiR₁₄R₁₅ and GeR₁₆R₁₇;
A₁ to A₃ are the same as or different from each other, and each independently any one selected from substituted or unsubstituted C₆-C₃₀ aromatic hydrocarbon rings, substituted or unsubstituted C₃-C₃₀ aliphatic hydrocarbon rings, substituted or unsubstituted C₂-C₃₀ aromatic heterocyclic rings, substituted or unsubstituted C₂-C₃₀ aliphatic heterocyclic rings, and substituted or unsubstituted cyclic groups in which a C₃-C₂₄ aliphatic ring and a C₅-C₂₄ aromatic ring are fused together;
R₁₁ to R₁₇ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted cyclic groups in which a C₃-C₃₀ aliphatic ring and a C₅-C₃₀ aromatic ring are fused together, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano and halogen;
R₁₁ to R₁₇ are optionally linked to the rings A₁ to A₃ to further form an alicyclic or aromatic monocyclic or polycyclic ring;
R₁₂ and R₁₃, R₁₄ and R₁₅, and R₁₆ and R₁₇ are optionally linked to each other to further form an alicyclic or aromatic monocyclic or polycyclic ring; and
the term ‘substituted’ in the ‘substituted or unsubstituted’ in [Formula 2] means being substituted with one or more substituents selected from the group consisting of deuterium, tritium, C₁-C₂₄ alkyl, C₁-C₂₄ haloalkyl, C₂-C₂₄ alkenyl, C₂-C₂₄ alkynyl, C₃-C₃₀ cycloalkyl, C₁-C₂₄ heteroalkyl, C₆-C₃₀ aryl, C₇-C₃₀ arylalkyl, C₇-C₃₀ alkylaryl, C₂-C₃₀ heteroaryl, C₂-C₃₀ heteroarylalkyl, cyclic groups in which a C₃-C₂₄ aliphatic ring and a C₅-C₂₄ aromatic ring are fused together, C₁-C₂₄ alkoxy, C₁-C₃₀ amine, C₁-C₃₀ silyl, C₁-C₃₀ germanium, C₆-C₂₄ aryloxy, C₆-C₂₄ arylthionyl, cyano, halogen, hydroxyl and nitro, and when there are two or more substituents, they are the same as or different from each other, and one or more hydrogen atoms in each of the substituents are optionally substituted with deuterium or tritium atoms.

12. The organic light emitting device according to claim 11, wherein A₁ to A₃ are the same as or different from each other, and each independently any one selected from substituted or unsubstituted C₆-C₃₀ aromatic hydrocarbon rings, substituted or unsubstituted C₆-C₃₀ aromatic heterocyclic rings and substituted or unsubstituted cyclic groups in which a C₃-C₂₄ aliphatic ring and a C₅-C₂₄ aromatic ring are fused together.

13. The organic light emitting device according to claim 1, wherein [Formula 1] is any one selected from compounds represented by the following formulae:

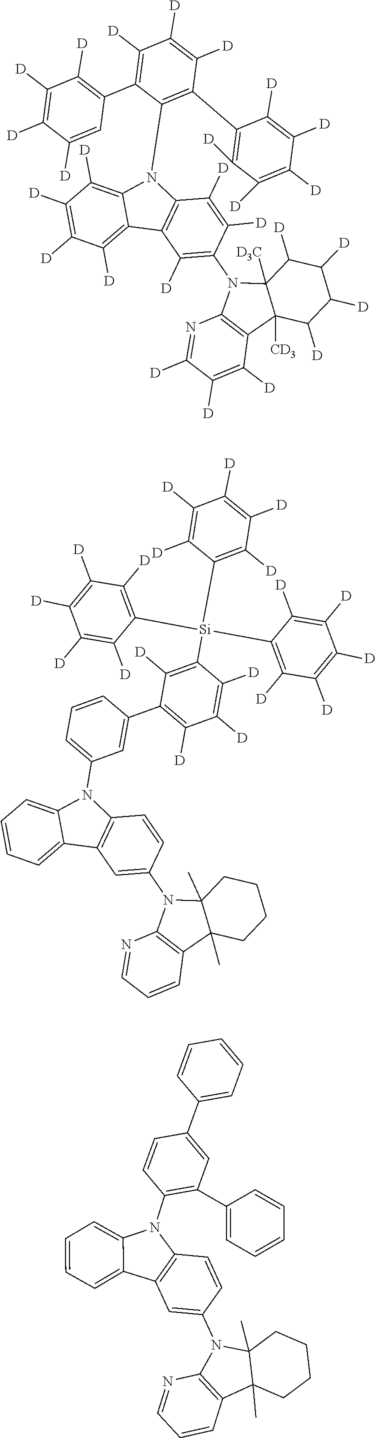

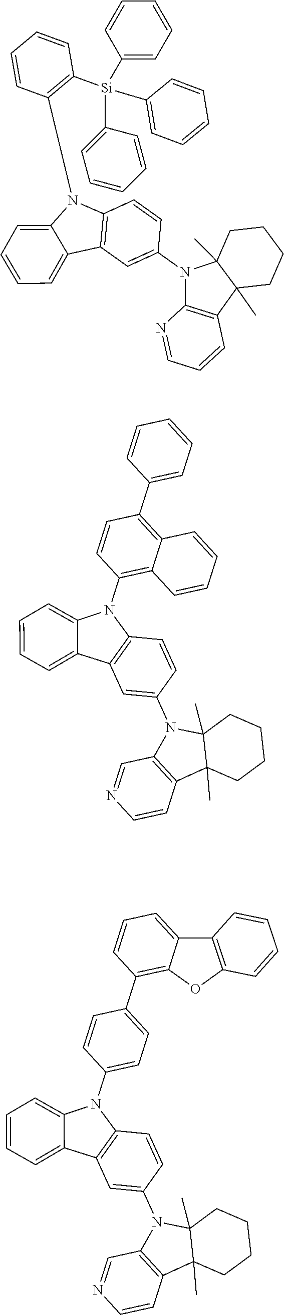 [SMILES: CC12CCCCC1(C)N(c1ccc3c(c1)c1ccccc1n3-c1ccccc1[Si](c1ccccc1)(c1ccccc1)c1ccccc1)c1ncccc12 ; CC12CCCCC1(C)N(c1ccc3c(c1)c1ccccc1n3-c1ccc(-c3ccccc3)c3ccccc13)c1cnccc12 ; CC12CCCCC1(C)N(c1ccc3c(c1)c1ccccc1n3-c1ccc(-c3cccc4c3oc3ccccc34)cc1)c1cnccc12]

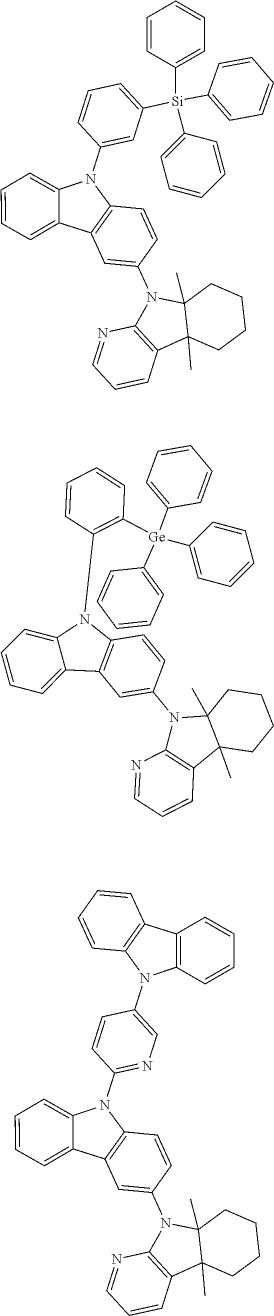

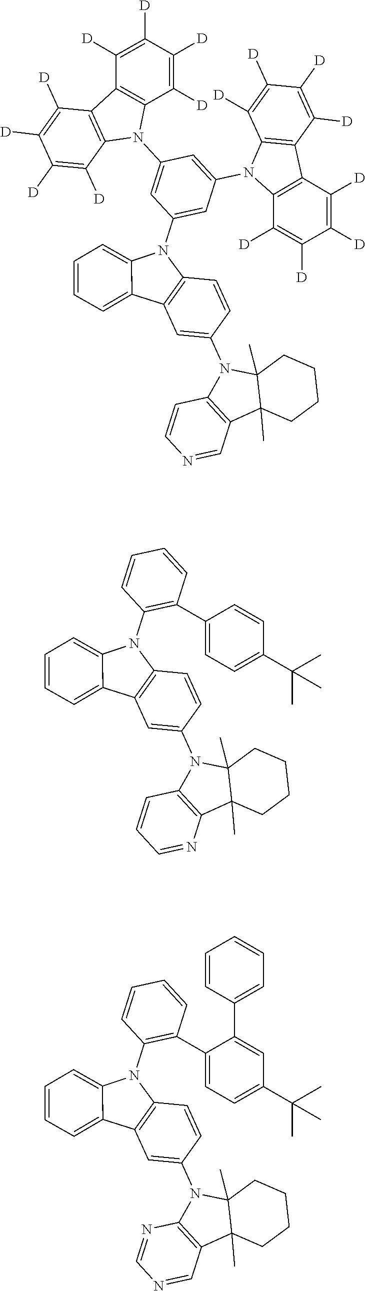 [SMILES: [2H]c1c([2H])c([2H])c2c(c1[2H])c1c([2H])c([2H])c([2H])c([2H])c1n2-c1cc(-n2c3ccccc3c3cc(N4c5ccncc5C5(C)CCCCC45C)ccc32)cc(-n2c3c([2H])c([2H])c([2H])c([2H])c3c3c([2H])c([2H])c([2H])c([2H])c32)c1 ; CC(C)(C)c1ccc(-c2ccccc2-n2c3ccccc3c3cc(N4c5cccnc5C5(C)CCCCC45C)ccc32)cc1 ; CC(C)(C)c1ccc(-c2ccccc2-n2c3ccccc3c3cc(N4c5ncncc5C5(C)CCCCC45C)ccc32)c(-c2ccccc2)c1]

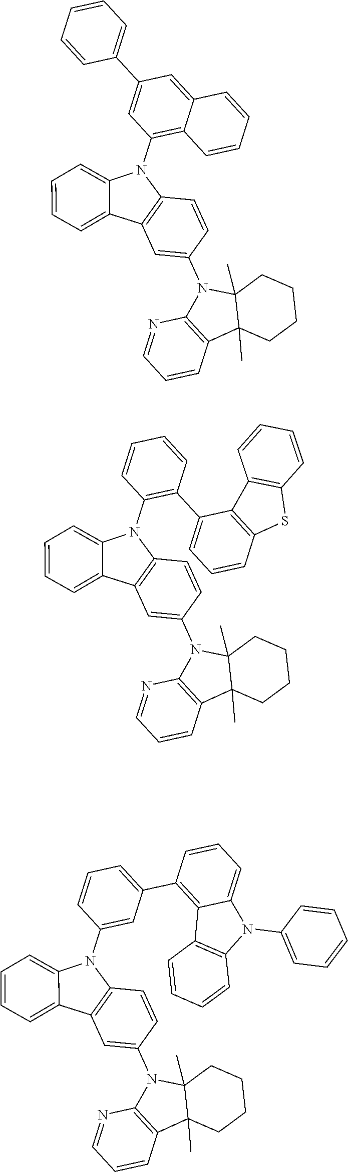 [SMILES: CC12CCCCC1(C)N(c1ccc3c(c1)c1ccccc1n3-c1cc(-c3ccccc3)cc3ccccc13)c1ncccc12 ; CC12CCCCC1(C)N(c1ccc3c(c1)c1ccccc1n3-c1ccccc1-c1cccc3sc4ccccc4c13)c1ncccc12 ; CC12CCCCC1(C)N(c1ccc3c(c1)c1ccccc1n3-c1cccc(-c3cccc4c3c3ccccc3n4-c3ccccc3)c1)c1ncccc12]

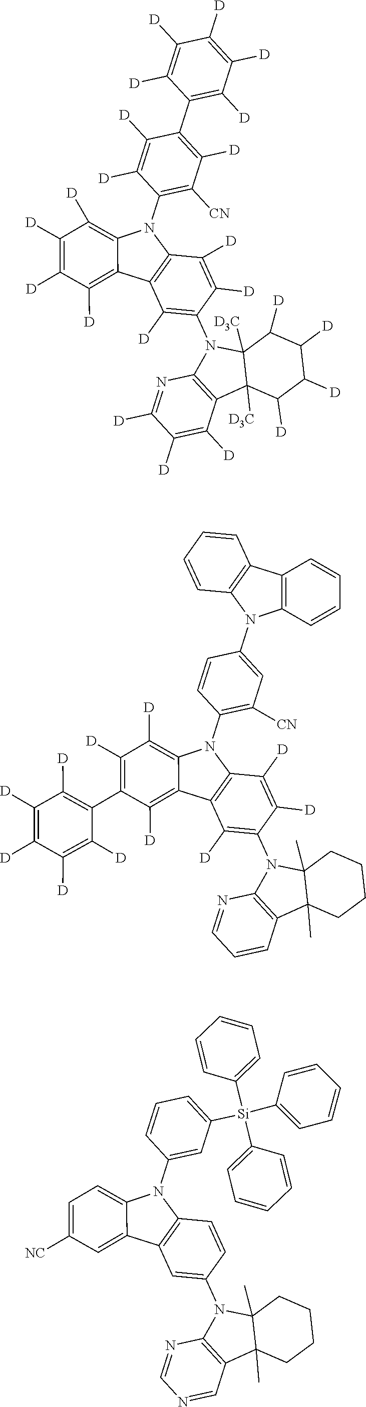 [SMILES: [2H]c1nc2c(c([2H])c1[2H])C1(C([2H])([2H])[2H])C([2H])C([2H])C([2H])C([2H])C1(C([2H])([2H])[2H])N2c1c([2H])c([2H])c2c(c1[2H])c1c([2H])c([2H])c([2H])c([2H])c1n2-c1c([2H])c([2H])c(-c2c([2H])c([2H])c([2H])c([2H])c2[2H])c([2H])c1C#N ; [2H]c1c([2H])c([2H])c(-c2c([2H])c([2H])c3c(c2[2H])c2c([2H])c(N4c5ncccc5C5(C)CCCCC45C)c([2H])c([2H])c2n3-c2ccc(-n3c4ccccc4c4ccccc43)cc2C#N)c([2H])c1[2H] ; CC12CCCCC1(C)N(c1ccc3c(c1)c1cc(C#N)ccc1n3-c1cccc([Si](c3ccccc3)(c3ccccc3)c3ccccc3)c1)c1ncncc12]

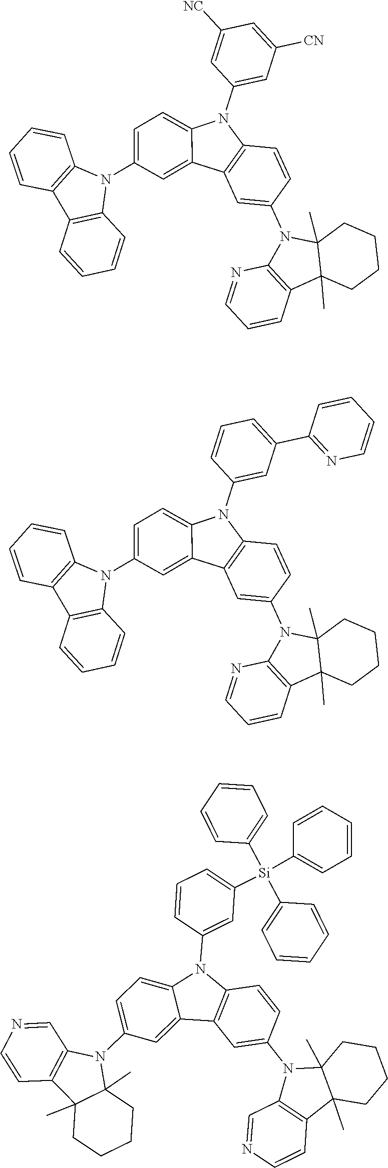 [SMILES: CC12CCCCC1(C)N(c1ccc3c(c1)c1cc(-n4c5ccccc5c5ccccc54)ccc1n3-c1cc(C#N)cc(C#N)c1)c1ncccc12 ; CC12CCCCC1(C)N(c1ccc3c(c1)c1cc(-n4c5ccccc5c5ccccc54)ccc1n3-c1cccc(-c3ccccn3)c1)c1ncccc12 ; CC12CCCCC1(C)N(c1ccc3c(c1)c1cc(N4c5cnccc5C5(C)CCCCC45C)ccc1n3-c1cccc([Si](c3ccccc3)(c3ccccc3)c3ccccc3)c1)c1cnccc12]

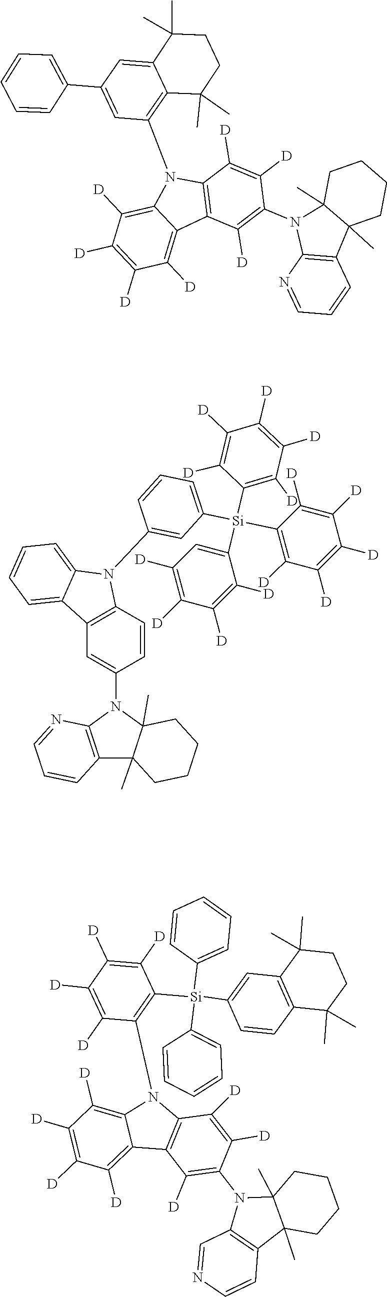

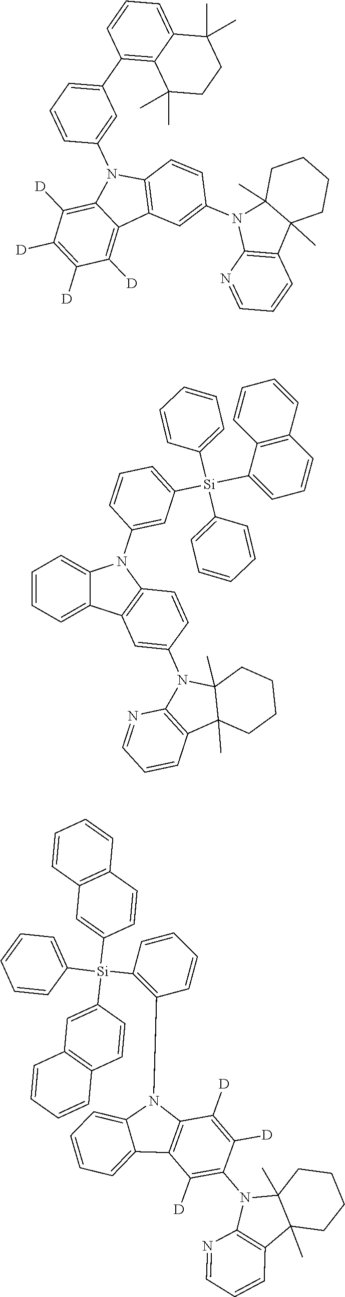 [SMILES: [2H]c1c([2H])c([2H])c2c(c1[2H])c1cc(N3c4ncccc4C4(C)CCCCC34C)ccc1n2-c1cccc(-c2cccc3c2C(C)(C)CCC3(C)C)c1 ; CC12CCCCC1(C)N(c1ccc3c(c1)c1ccccc1n3-c1cccc([Si](c3ccccc3)(c3ccccc3)c3cccc4ccccc34)c1)c1ncccc12 ; [2H]c1c(N2c3ncccc3C3(C)CCCCC23C)c([2H])c2c3ccccc3n(-c3ccccc3[Si](c3ccccc3)(c3ccc4ccccc4c3)c3ccc4ccccc4c3)c2c1[2H]]

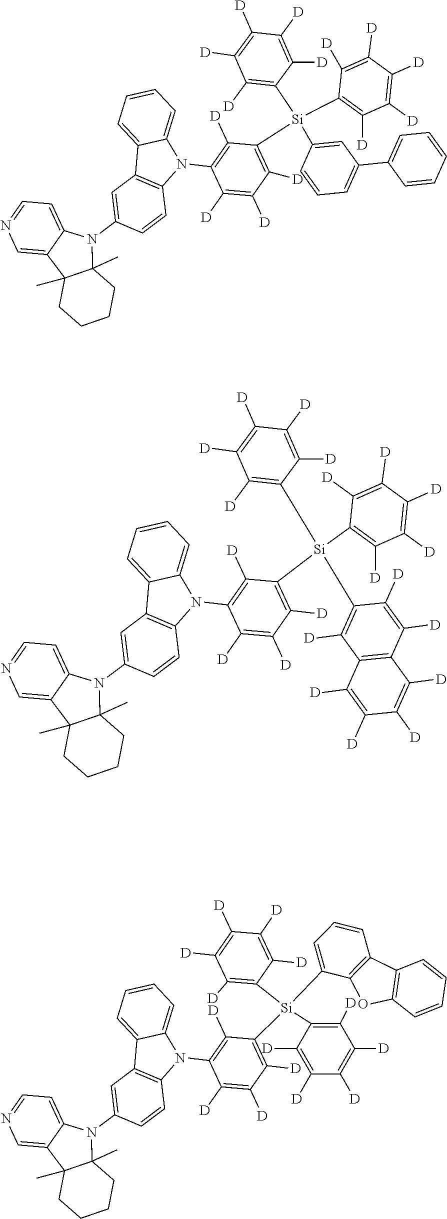

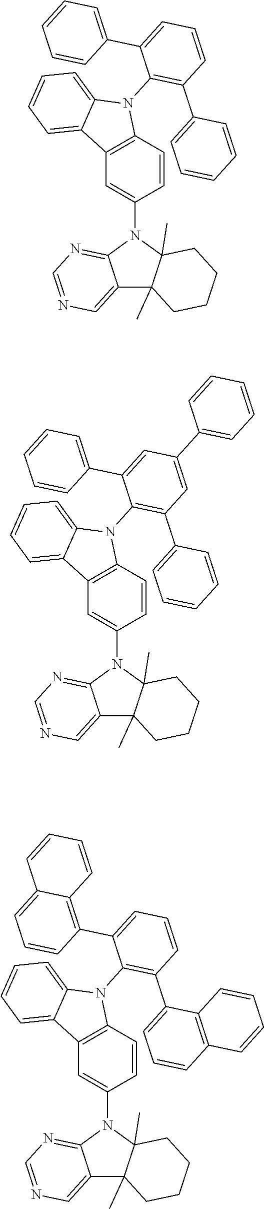 [SMILES: CC12CCCCC1(C)N(c1ccc3c(c1)c1ccccc1n3-c1c(-c3ccccc3)cccc1-c1ccccc1)c1ncncc12 ; CC12CCCCC1(C)N(c1ccc3c(c1)c1ccccc1n3-c1c(-c3ccccc3)cc(-c3ccccc3)cc1-c1ccccc1)c1ncncc12 ; CC12CCCCC1(C)N(c1ccc3c(c1)c1ccccc1n3-c1c(-c3cccc4ccccc34)cccc1-c1cccc3ccccc13)c1ncncc12]

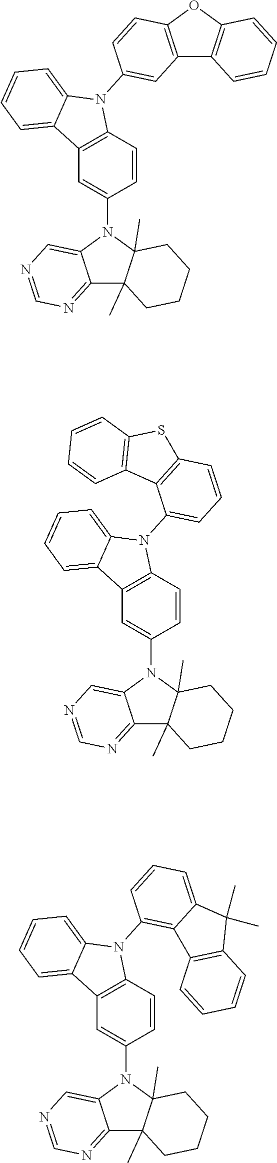 [SMILES: CC12CCCCC1(C)N(c1ccc3c(c1)c1ccccc1n3-c1ccc3oc4ccccc4c3c1)c1cncnc12 ; CC12CCCCC1(C)N(c1ccc3c(c1)c1ccccc1n3-c1cccc3sc4ccccc4c13)c1cncnc12 ; CC1(C)c2ccccc2-c2c(-n3c4ccccc4c4cc(N5c6cncnc6C6(C)CCCCC56C)ccc43)cccc21]

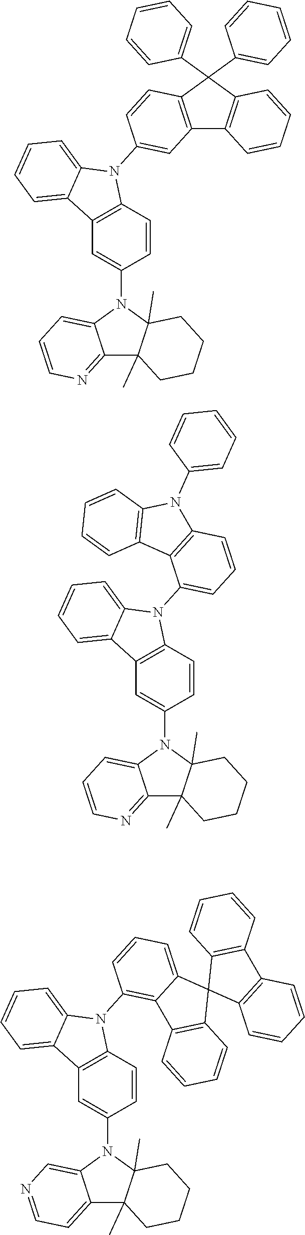

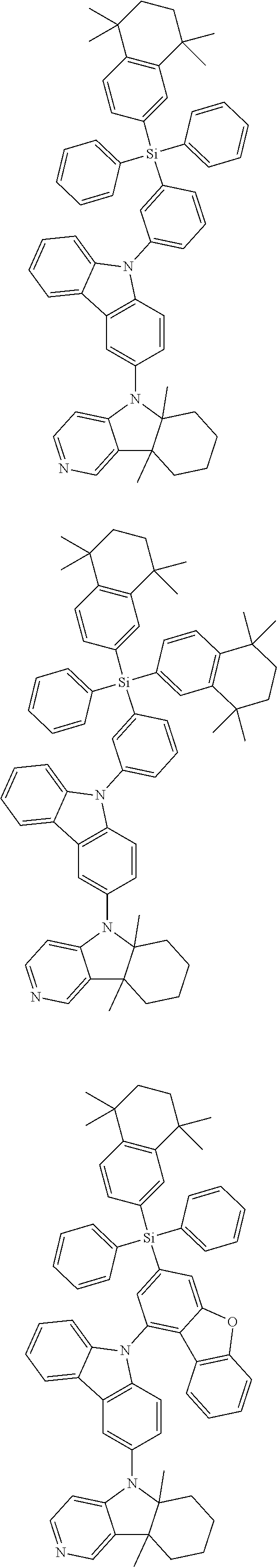 [SMILES: CC1(C)CCC(C)(C)c2cc([Si](c3ccccc3)(c3ccccc3)c3cccc(-n4c5ccccc5c5cc(N6c7ccncc7C7(C)CCCCC67C)ccc54)c3)ccc21 ; CC1(C)CCC(C)(C)c2cc([Si](c3ccccc3)(c3cccc(-n4c5ccccc5c5cc(N6c7ccncc7C7(C)CCCCC67C)ccc54)c3)c3ccc4c(c3)C(C)(C)CCC4(C)C)ccc21 ; CC1(C)CCC(C)(C)c2cc([Si](c3ccccc3)(c3ccccc3)c3cc(-n4c5ccccc5c5cc(N6c7ccncc7C7(C)CCCCC67C)ccc54)c4c(c3)oc3ccccc34)ccc21]

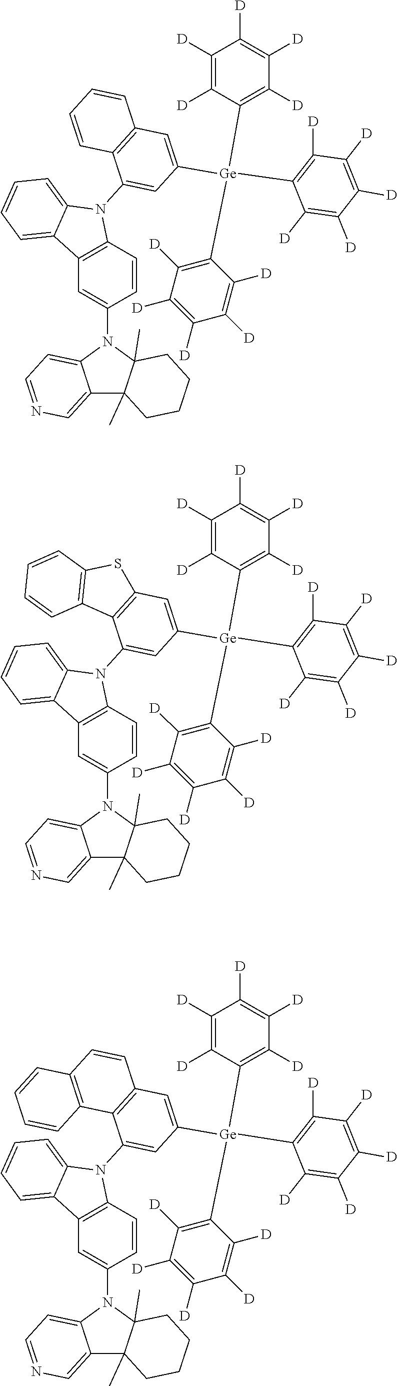

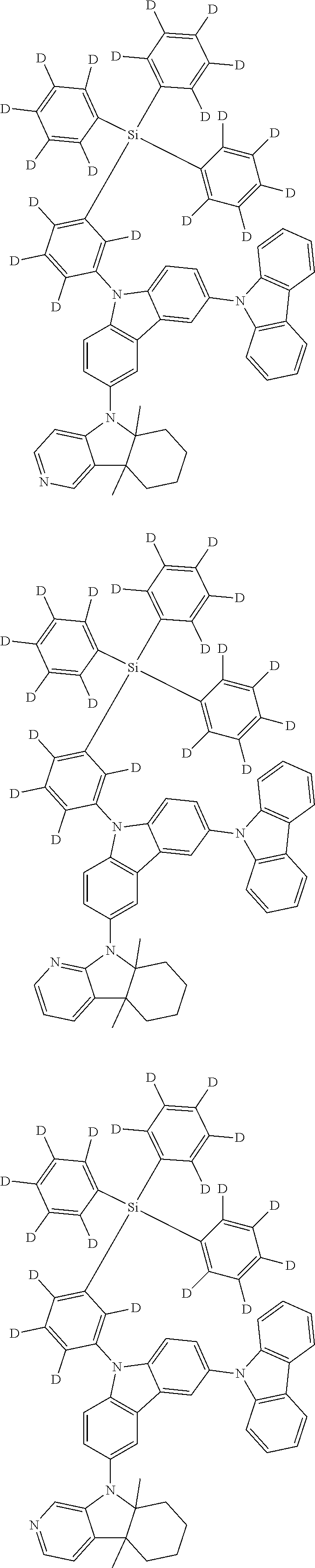

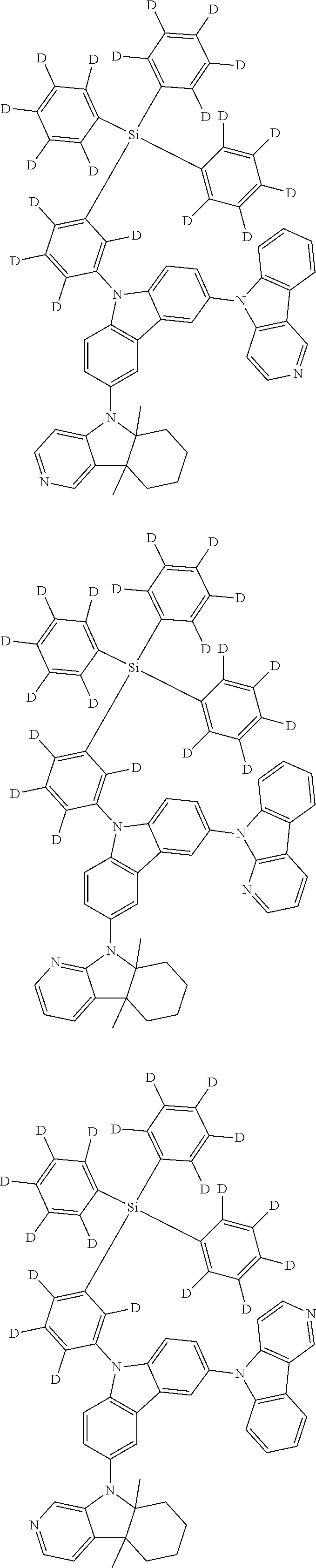

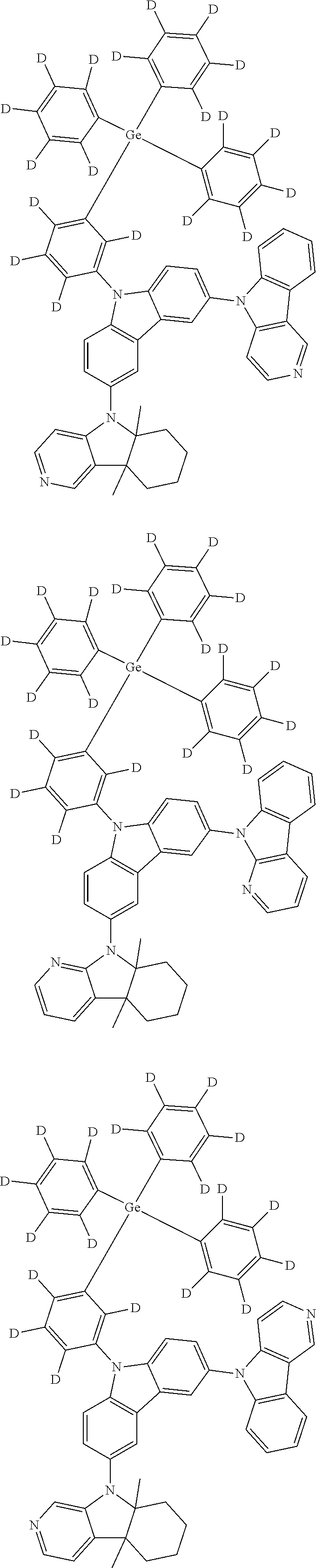

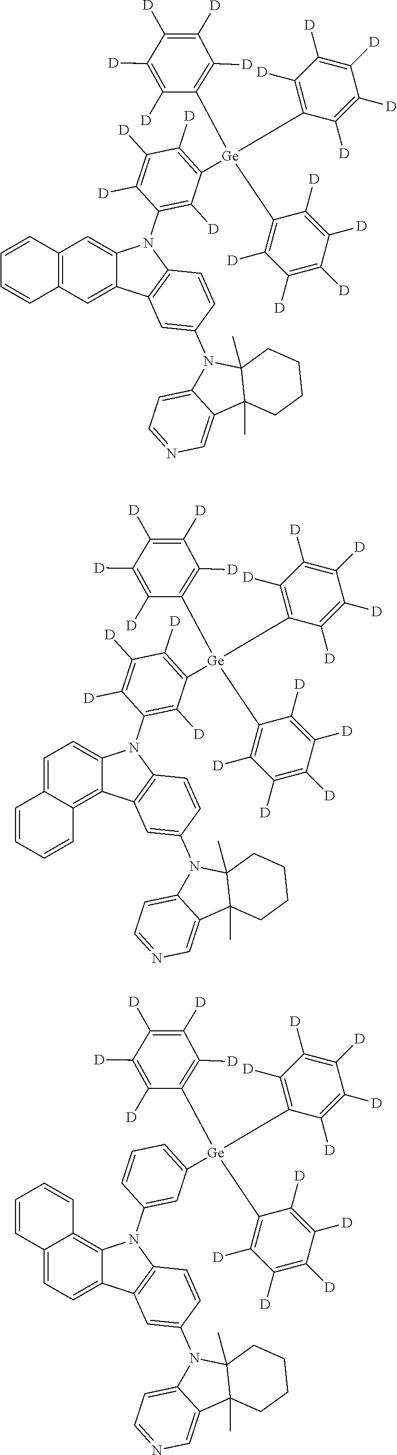

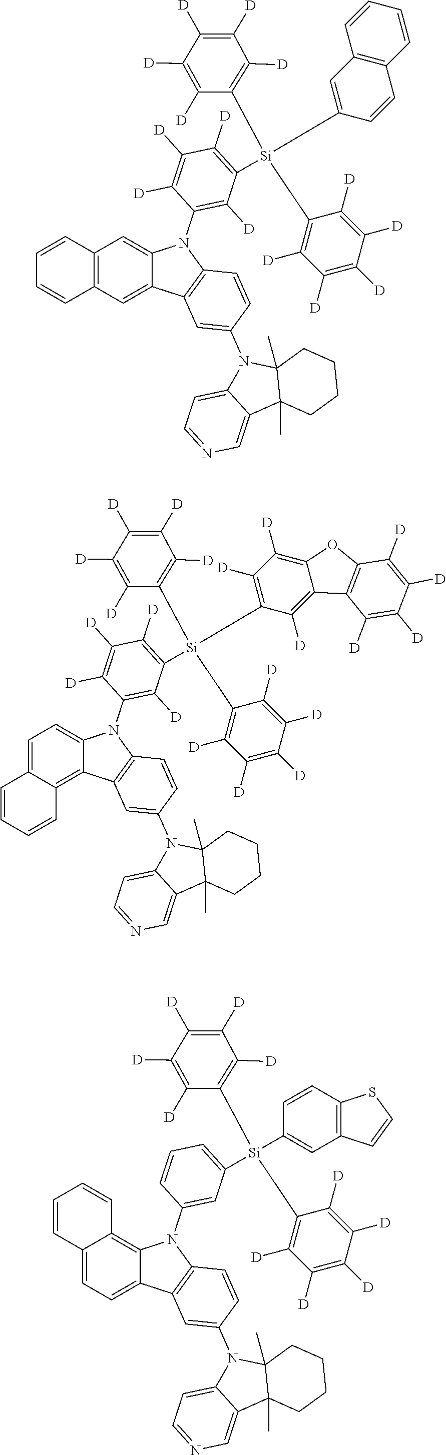

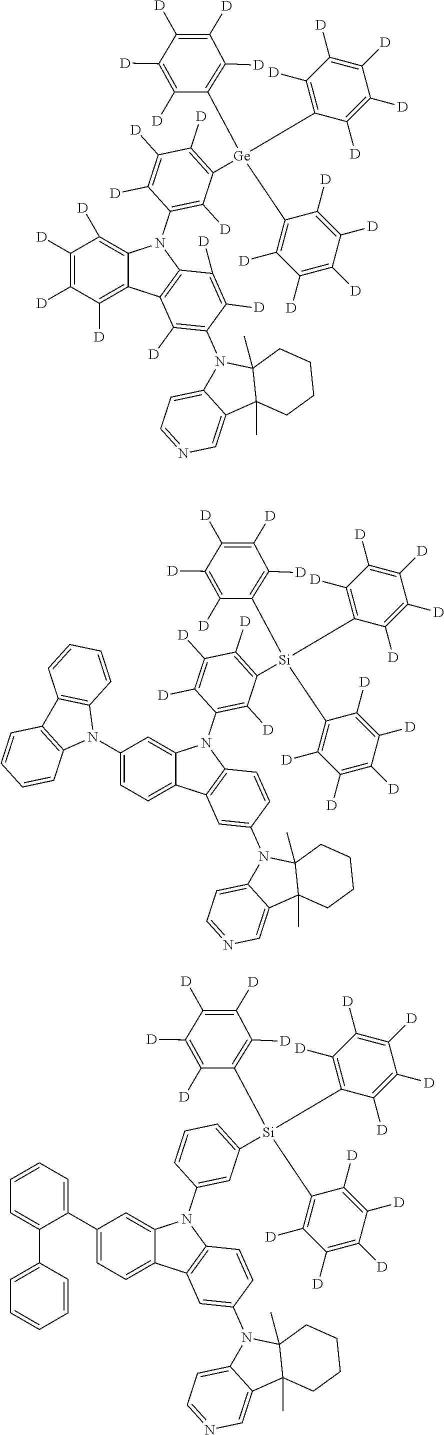

14. The organic light emitting device according to claim 1, wherein the organic light emitting device is used in any one device selected from flat panel displays, flexible displays, monochromatic or white flat panel lighting systems, monochromatic or white flexible lighting systems, displays for automotives and aircraft, and displays for virtual or augmented reality.

15. An organic compound represented by the following [Formula 1]: 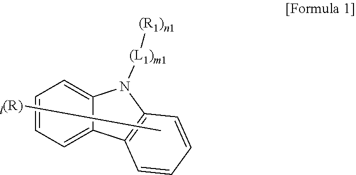
wherein, in [Formula 1],
L₁s are the same as or different from each other, and each independently a single bond, or any one selected from substituted or unsubstituted C₆-C₃₀ arylene, substituted or unsubstituted C₂-C₃₀ heteroarylene, and substituted or unsubstituted divalent cyclic groups in which a C₃-C₂₄ aliphatic ring and a C₅-C₂₄ aromatic ring are fused together;
R and R₁ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted cyclic groups in which a C₃-C₃₀ aliphatic ring and a C₅-C₃₀ aromatic ring are fused together, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano and halogen;
however, at least one of Rs of [Formula 1] is [Structural Formula 1], 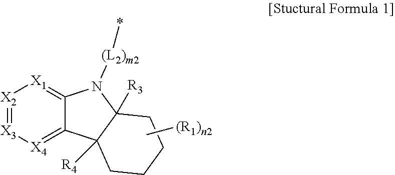
in [Structural Formula 1],
‘*’ means a bonding site linked to a carbon atom (C) in the carbazole of [Formula 1];
X₁ to X₄ are the same as or different from each other and each independently N or CR₅, and at least one of X₁ to X₄ is N;
L₂s are the same as or different from each other, and each independently a single bond, or any one selected from substituted or unsubstituted C₆-C₃₀ arylene, substituted or unsubstituted C₂-C₃₀ heteroarylene, and substituted or unsubstituted divalent cyclic groups in which a C₃-C₂₄ aliphatic ring and a C₅-C₂₄ aromatic ring are fused together;
R₂ and R₅ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, substituted or unsubstituted cyclic groups in which a C₃-C₃₀ aliphatic ring and a C₅-C₃₀ aromatic ring are fused together, substituted or unsubstituted C₁-C₃₀ alkoxy, substituted or unsubstituted C₆-C₃₀ aryloxy, substituted or unsubstituted C₁-C₃₀ alkylthioxy, substituted or unsubstituted C₅-C₃₀ arylthioxy, substituted or unsubstituted amine, substituted or unsubstituted silyl, substituted or unsubstituted germanium, nitro, cyano and halogen;
R₃ and R₄ are the same as or different from each other, and each independently any one selected from hydrogen, deuterium, tritium, substituted or unsubstituted C₁-C₃₀ alkyl, substituted or unsubstituted C₁-C₃₀ haloalkyl, substituted or unsubstituted C₂-C₃₀ alkynyl, substituted or unsubstituted C₂-C₃₀ alkenyl, substituted or unsubstituted C₆-C₅₀ aryl, substituted or unsubstituted C₃-C₅₀ cycloalkyl, substituted or unsubstituted C₂-C₅₀ heterocycloalkyl, substituted or unsubstituted C₂-C₅₀ heteroaryl, and substituted or unsubstituted cyclic groups in which a C₃-C₃₀ aliphatic ring and a C₅-C₃₀ aromatic ring are fused together;
n₁ is an integer of 1 to 3, and when it is an integer of 2 or greater, R₁s are the same as or different from each other;
n₂ is an integer of 4, and R₂s are the same as or different from each other;
m₁ and m₂ are each independently an integer of 1 to 3, and when they are integers of 2 or greater, L₁s and L₂s are the same as or different from each other;
1 is an integer of 8, and Rs except for the part linked to [Structural Formula 1] are the same as or different from each other;
the plurality of adjacent Rs and R₅s are optionally linked to each other to further form an alicyclic or aromatic monocyclic or polycyclic ring; and
the term ‘substituted’ in the ‘substituted or unsubstituted’ in [Formula 1] and [Structural Formula 1] means being substituted with one or more substituents selected from the group consisting of deuterium, tritium, C₁-C₂₄ alkyl, C₁-C₂₄ haloalkyl, C₂-C₂₄ alkenyl, C₂-C₂₄ alkynyl, C₃-C₃₀ cycloalkyl, C₁-C₂₄ heteroalkyl, C₆-C₃₀ aryl, C₇-C₃₀ arylalkyl, C₇-C₃₀ alkylaryl, C₂-C₃₀ heteroaryl, C₂-C₃₀ heteroarylalkyl, cyclic groups in which a C₃-C₂₄ aliphatic ring and a C₅-C₂₄ aromatic ring are fused together, C₁-C₂₄ alkoxy, C₁-C₃₀ amine, C₁-C₃₀ silyl, C₁-C₃₀ germanium, C₆-C₂₄ aryloxy, C₆-C₂₄ arylthionyl, cyano, halogen, hydroxyl and nitro, and when there are two or more substituents, they are the same as or different from each other, and one or more hydrogen atoms in each of the substituents are optionally substituted with deuterium or tritium atoms.
